(19) United States
(12) Patent Application Publication
(10) Pub. No.: US 2025/0057934 A1
(43) Pub. Date: Feb. 20, 2025

(54) STABLE EMULSIONS OF ANTIGENS

(71) Applicant: INTERVET INC., Madison, NJ (US)

(72) Inventors: Martin PIEST, Afferden (NL); Marc THEELEN, Lomm (NL); Franciscus Gerardus Antonios MANDERS, Helden (NL); Maarten Hendrik WITVLIET, Oostrum (NL)

(73) Assignee: INTERVET INC., Madison, NJ (US)

(21) Appl. No.: 18/723,268

(22) PCT Filed: Dec. 23, 2022
(86) PCT No.: PCT/EP2022/087692

§ 371 Date: Jun. 21, 2024

(30) Foreign Application Priority Data
Dec. 24, 2021 (EP) 21217746.3

(51) Int. Cl. A61K 39/135; A61K 9/107; A61K 39/00; A61K 39/39; B82Y 5/00; C12N 7/00

(52) U.S. Cl. CPC A61K 39/135; A61K 9/1075; A61K 39/39; C12N 7/00; A61K 2039/54; A61K 2039/55566; B82Y 5/00; C12N 2770/32134

(57) ABSTRACT

The present invention relates to the field of vaccinology, more specifically of veterinary vaccinology. In particular, the invention relates to an adjuvant composition comprising an emulsion of water, tocopherol or a pharmaceutically acceptable ester thereof, and a polyethoxyethylene cetostearyl ether as an emulsifier. Said adjuvant composition can be used for formulating a vaccine, particularly an emulsion vaccine, comprising a bacterial, parasitic, or viral antigen. The resulting vaccine composition can be used in a method of protecting a human or animal target against infection and/or disease caused by a pathogen, particularly caused by a bacterium, parasite or virus. The invention further relates to methods for the manufacture of such adjuvant compositions and for the manufacture of such vaccine composition.

### FIELD OF THE INVENTION

The present invention relates to the field of vaccinology, more specifically of veterinary vaccinology. In particular, the invention relates to an adjuvant composition comprising an emulsion of water, tocopherol or a pharmaceutically acceptable ester thereof, and a polyethoxyethylene cetostearyl ether as an emulsifier. Said adjuvant composition can be used for formulating a vaccine, particularly an emulsion vaccine, including a bacterial, parasitic and/or viral antigen. The resulting vaccine composition can be used in a method of protecting a human or animal target against infection and/or disease caused by a pathogen, particularly caused by a bacterium, parasite or virus. The invention further relates to methods for the manufacture of such adjuvant compositions and for the manufacture of such vaccine composition.

### INTRODUCTION

Infections by pathogenic bacteria, parasites or viruses and their resulting diseases are known since the beginning of civilisation, affecting both humans and animals with their often serious effects on health and well-being.

Since the mid-20^{th} century, bacterial infections can be effectively treated with antibiotic drugs, although build-up of resistance is a constant threat. The use of antibiotics in animal husbandry in the agricultural sector, is a special situation: on the one hand there is an urgent need for treatment because of a high infection-pressure resulting from the manner and conditions under which animals are often kept. On the other hand the generalised non-therapeutic use of antibiotics, such as in animal feed, is now recognised as one of the causes for increase of resistance in bacteria that are also relevant to human health. As a result such generalised use of antibiotics is being phased-out in more and more countries.

A similar situation applies to the prophylactic use of anti-parasitic drugs.

In the case of viral infections the situation is even more challenging since the use of antiviral drugs is often too expensive for veterinary purposes.

Next to improvements in farm-management, the best alternative for fighting pathogenic infection and disease in a sustainable way, is by vaccination. Bacterial, parasitic and viral vaccines, both for humans and for animals, are well known for over a century, and are available in many forms. Such vaccines can be live, i.e. contain replicative (attenuated) bacteria, parasites or viruses, or non-live, i.e. comprise inactivated bacteria, parasites or viruses, or one or more of their components.

Vaccines that comprise non-live (i.e. non-replicating) antigens, often require an adjuvant to provide an immune-stimulation for the non-live antigen. In veterinary vaccines, a great variety of compounds can be used as adjuvant, for example: mineral oil e.g. Bayol™ or Markol™ Montanide™ or paraffin oil; non-mineral oil such as squalene, squalane, or vegetable oils, e.g. ethyl oleate; aluminium salts, e.g. aluminium hydroxide, or aluminium phosphate; peptides such as dimethylglycine, or tuftsin; bacterial cell-wall components, such as lipid A and muramyldipeptide; (synthetic) polymers, such as pluronics, dextranes, carbomeres, pyran, or saponin; cytokines; and stimulators of toll-like receptors such as immunostimulatory oligodeoxynucleotides containing non-methylated CpG groups; etc.

For ease of administration, and for enhanced adjuvant effect, an oil adjuvant can be emulsified with an antigen that is in an aqueous phase, to form an emulsion which in turn can be used for the preparation of a vaccine. In such an emulsion one liquid phase is dispersed in another, typically as a water-in-oil (W/O) or as an oil-in-water (O/W) type emulsion. The choice for one or the other type of emulsion can be based e.g. on the type of immune-response that is desired.

To generate and maintain such an emulsion requires the input of both mechanical—as well as chemical energy: the separate liquids are mixed in an appropriate device using certain levels of shear-force, pressure, and temperature to disperse one phase into another. Input of chemical energy is provided by the use of an emulsifier (also: surfactant) which stabilises the dispersed phase by taking position at the interphase of water and oil. A vaccine emulsion can be made up of one or more adjuvants, with one or more emulsifiers.

A large number of emulsifiers for use in emulsion vaccines is available, and more are constantly being developed.

Examples of combinations of adjuvants and emulsifiers as used in commercial veterinary vaccines are: Amphigen® (Zoetis), containing a light mineral oil with lecithin as emulsifier; Xsolve® (previously called: Microsol-Diluvac Forte®, MSD Animal health), which contains a combination of the adjuvants light mineral oil and vitamin E-acetate, with the emulsifier Tween® 80 (Polysorbate 80 or polyoxyethylene sorbitan mono-oleate); and MetaStim® (Zoetis), comprising squalane, Pluronic® (a non-ionic tri-block copolymer of blocks of polyoxyethylene and polyoxypropylene), and Tween 80; Fortasol® (a.k.a. Diluvac Forte®) comprises Vitamin-E acetate and Tween 80; SP-oil™ (Pluronic™, squalane, and Tween); and ASO3™ (GSK), which contains 2.1% w/v squalene and 2.4% w/v vitamin E, with 1.0% w/v Tween 80 as emulsifier.

An emulsion for use as a vaccine should be stable and not ‘break’, meaning that the type, size, and number of the droplets of the dispersed phase should not change too much over time, which could eventually lead to reduction of dispersion and ultimately to a complete phase separation.

Maintaining the stability of the emulsion is important to guarantee the use and efficacy of an emulsion vaccine during its registered shelf-life. Effects of gravity over time, such as sedimentation or creaming are harmless and can readily be reversed by shaking by hand. However actual breaking of an emulsion is irreversible, and the resulting sub-optimal distribution of the phases may lead to incorrect dosing, to safety issues, and can affect the immunological potency of the vaccine antigen(s).

Next to the paramount requirements for vaccines to be safe and efficacious, there are some special requirements for vaccines used in animal husbandry. These refer to aspects of ease of use, and especially to costs. This because the production of animal protein is typically a high volume-low margin enterprise. For these reasons, veterinary vaccines will often be directed at several diseases or pathogens at once, by containing several different antigens in a single vaccine formulation. This is favourable to reduce stress for a target animal from prevention of the need for repeated treatments, as well as to reduce labour costs for the administration.

Currently, oil-adjuvanted emulsion vaccines with bacterial, parasitic and/or viral antigens are commercially available for a large number of pathogens, and for all the major animal target groups: swine, cattle, sheep, poultry, companion animals (e.g. cats, dogs, horses), and fish.

However not all desired selections of antigens can be formulated into stable emulsion vaccines. This is because of a further consequence of the need to make vaccines for use in agriculture affordable, namely: that such vaccines will typically not contain expensive pure components (e.g. recombinantly expressed subunits), and cannot be produced using elaborate purification techniques (e.g. column chromatography). In practice this means that non-live antigens comprised in such vaccines, will typically be relatively impure and of somewhat undefined composition. This applies especially when compared to more purified vaccine antigens in vaccine products of higher market value such as for use in companion animals (cats, dogs, and horses), or even as for use in humans. Alternatively, the specific protective antigen may not be known, so that a crude antigen preparation is the only way to comprise the necessary antigens. Non-live bacterial, parasitic, or viral vaccines for use in agriculture will therefore typically comprise little purified antigens derived from, for example, an inactivated bacterial, parasitic or viral culture, or from extracts or fractions of such a culture. Such rather crude antigens can be based on inactivated bacteria or parasites or on inactivated viruses, perhaps washed or concentrated once; or antigen based on bacterial-, parasitic- or viral fractions, such as bacterial cells, parasites or viruses that were lysed or disrupted. As a consequence, these crude antigen preparations may contain undefined or unintended impurities which can have an effect on the safety, the efficacy, or the stability of a (combination) vaccine. This is why vaccines as part of their development process, must undergo rigorous testing for safety, efficacy and stability, before they can receive a marketing authorisation from governmental- or regulatory authorities to place such vaccine on the market as a commercial product.

The unspecified components of a non-live antigen preparation are not necessarily unwanted, as they may act as a further adjuvant and in this way provide an aspecific boost to the immune response. Also, potentially disturbing factors of a biological nature would be expected to become inactivated during the preparation of the non-live bacterial, parasitic or viral antigens. Nevertheless unwanted effects on safety, efficacy or stability of a vaccine can be observed during the development of an emulsion-based vaccine comprising a crude antigen preparation.

Another obstacle to overcome in making adjuvanted vaccines, is to prevent an interaction between the various vaccine components that would negatively influence the immune response or the vaccine's safety or stability. Such interaction may for instance occur between the antigens themselves, e.g. because some are quite crude products. Also, the adjuvant may interfere with, or even damage a vaccine antigen. It is thus difficult to develop an adjuvanted vaccine which induces an effective immune-response against one or more antigens especially for complex combinations relating to antigens from multiple species of pathogens.

Further the adjuvanted vaccine should be safe upon use in animals, i.e. not produce significant side reactions such as fever, local swelling, loss of appetite, etc. Also more practical properties are relevant: the emulsion vaccine should ideally be capable of economic production, be sufficiently stable during formulation and storage, and allow potency testing methods for each antigen, in the presence of the other antigens.

Some of the most prominent diseases affecting swine from a young age onwards are caused by bacteria such as: Mycoplasma hyopneumoniae and Lawsonia intracellularis; and by viruses such as porcine circovirus type 2 (PCV2), and porcine reproductive and respiratory syndrome virus (PRRSV).

Mycoplasma hyopneumoniae (Mhyo) is the primary agent causing (porcine) enzootic pneumonia, a chronic respiratory disease in swine, occurring worldwide. Especially young piglets are vulnerable to this highly contagious disease. The bacterium is relatively small, lacks a cell wall, and belongs to the genus Mollicutes. These bacteria live a parasitic lifestyle on or inside host cells.

Pulmonary disease from Mhyo is largely an immune-mediated pathology leading to consolidated pneumonia. The bacterium colonizes and damages the pulmonary ciliated epithelium, leading to loss of ciliary activity. Depending on housing conditions and environmental stress, the most problematic consequence of this disease is that it predisposes for different secondary infections of the porcine respiratory system, e.g. by other bacterial and viral pathogens. This gives rise to the so called: Porcine Respiratory Disease Complex (PRDC), displaying severe lung lesions. Next to discomfort to the animal, enzootic pneumonia and PRDC cause important economic losses to the swine industry due to reduced performance in growth rate and feed conversion ratio, as well as through costs for veterinary care and antibiotics use.

Lawsonia intracellularis causes proliferative enteropathy, also known as ileitis, which is a common enteric disease of post-weaning pigs worldwide. The characteristic lesion is a proliferation of immature enterocytes in the ileal intestinal crypts, which cells contain the causative bacteria. Clearance of the bacteria from the enterocytes leads to resolution of the associated proliferative lesions. Histologic lesions can be confirmed as Lawsonia-positive by visualization of 1.5-2.5 μm long, vibrioid shaped bacteria in the enterocytes, but also within intestinal macrophages. The bacteria can be detected via PCR in clinical or in subclinical cases. Clinical cases are usually present in the grower-finisher period.

L. intracellularis bacteria are obligate intracellular, and non-motile gram-negative bacilli, from the Desulfovibrionaceae family.

Porcine circovirus type 2 (PCV2) is linked to the post-weaning multisystemic wasting syndrome (PMWS) observed in young pigs. The clinical signs and pathology were published in 1996, and include progressive wasting, dyspnoea, tachypnoea, and occasionally icterus and jaundice. The new agent was called PCV2 as being different from the known PCV that was a natural contaminant of PK-15 cells.

PCV2 is a very small non-enveloped virus of the Circovirus genus. It contains a circular single stranded DNA genome with two major genes. The ORF2 gene encodes the viral capsid protein of about 233 amino acids. Recombinantly expressed PCV2 ORF2 proteins form virus-like particles which are highly effective as a subunit vaccine.

Porcine reproductive and respiratory syndrome virus (PRRSV) was first reported in 1987, and in the early 1990's had become pandemic. It is a small, enveloped RNA virus of the Arterivirus genus, which contains a single-stranded, positive-sense, RNA genome. The virus causes significant losses in the swine industry due to reproductive disorders and growth retardation. Like Mhyo, PRRSV plays a significant role in the multifactorial PRDC. Clinical symptoms are abortions and stillborn or mummified foetuses, and cyanosis of the ear and vulva. In neonatal pigs, the virus causes respiratory distress, with increased susceptibility to secondary respiratory infections such as Glasser's disease (caused by Haemophilus parasuis). However subclinical infections are also common. The virus is quite variable: next to the European variant (type 1) and the North American variant (type 2), there is now a third genotype: a highly pathogenic variant which emerged in China in 2000 and is now causing severe disease in swine in Asia.

Commercial vaccines against each of these pathogens exist.

To limit stress to the animals and cost and labour for the caretakers, some swine vaccines have been prepared as combination vaccine.

WO 2018/115435 describes a combination vaccine for swine comprising non-live antigen from PCV2 and live PRRSV. The vaccine is an oil-in-water emulsion comprising squalane and vitamin E-acetate.

WO 2021/048338 describes a combination vaccine for swine for protection against a pathogenic infection with PCV2 and Mhyo comprising non-live immunogen of PCV2 and non-live immunogen of Mhyo. The vaccine is an oil-in-water emulsion comprising squalane, vitamin E-acetate and silica.

There remains an interest in the field of veterinary vaccinology for effective vaccines, particularly for effective vaccines that are safe and effective across species.

FMD which is a highly contagious and devastating disease of cloven-hoofed animals worldwide has had significant economic impact. The disease is characterized by fever, lameness, lymphopenia and the appearance of vesicular lesions on the mouth, tongue, nose, feet and teats and is controlled by inhibition of susceptible animal movement, slaughter of infected animals and vaccination. In most countries, animals are immunized with inactivated whole virus vaccines to control the spread of foot-and-mouth disease virus (FMDV). However, the vaccine technology used comes with efficacy concerns, particularly with respect to cell-mediated immunity. In an attempt to address these concerns alternative vaccines are currently developed such as recombinant protein and peptide vaccines, empty capsid vaccines and genetically engineered inactivated vaccines. Several kinds of vaccine adjuvants have been studied for their potency to promote immune response to FMDV vaccines. These adjuvants include mineral oil, saponins (Quil-A), Toll-like receptor (TLR) ligands, cytokines, liposomes and so on (Y. Cao, 2014, Expert Rev. Vaccines, vol. 13, p. 1377-1385).

To address this issue, many efforts are currently devoted to the development of effective vaccines by combining the application of protective antigens together with the search for effective adjuvants that maximize the immunogenicity towards a desired immune response.

Cao (supra) provides a review relating to the action mechanisms and immunostimulatory effects of adjuvants both traditional and currently under development for FMD vaccines. In the following, some of the most promising approaches are briefly discussed.

The traditional FMD inactivated whole virus vaccines are often formulated in aqueous Al(OH)₃ and saponins, or in oil-based adjuvants. Of these, the Al(OH)₃/saponin-based vaccines are not ideal for use in pigs as their protective efficacy is low in this species.

In contrast thereto, W/O/W emulsion vaccines based on Montanide ISA-206 oil adjuvant (a mineral oil-based adjuvant containing esters of octadecenoic acid and anhydromannitol in an oily solution) with an inactivated antigen, are preferred for FMD prevention as they can be used to protect all susceptible species and are ideal for emergency vaccination. Also, the oil-adjuvanted vaccines generate higher and longer lasting immune responses than the Al(OH)₃-adjuvanted vaccines. Newly developed mineral oil-based adjuvant Montanide ISA-201 is suggested as an alternative as it seems to induce earlier and higher neutralizing antibody responses, higher cellular immunity and protective efficacy in cattle, as compared to ISA-206.

The real challenge for FMD vaccines is to find an adjuvant that works across species. For ruminants most adjuvated vaccines use W/O emulsions, and for swine typically O/W emulsions. The current gold standard in FMDV vaccines, are W/O/W emulsion vaccines based on Montanide ISA-206. However that only provides a partial and complex solution to the problem of a cross-species FMDV vaccine composition: as a ‘double emulsion’, or more precisely: a reversible emulsion, these have several drawbacks. An important one is the fact that the emulsion reverses above 32° C., meaning that vaccines should be kept refrigerated as much as possible and that temperatures at or above 32° C. should be avoided. This can be a challenge in countries where ‘cold chain’ logistics cannot always be maintained. Another drawback for use is that this emulsions is hard to inject (has poor syringeability), due to its composition. A further point of concern is the sensitivity of the formulation for high antigen payloads which can occur in vaccines containing multiple FMDV antigens. While that may be necessary to cover all the strains circulating in the field, however that has been observed to break multivalent FMDV vaccine compositions based on Montanide ISA-206 emulsions.

As a further alternative, immunostimulating complexes (ISCOMs) composed of saponin (such as Quil-A), cholesterol, phospholipid and antigen have been described. ISCOMs have been shown to elicit high-titred long-lasting antibodies and strong helper- and cytotoxic T lymphocyte responses. ISCOMs have been described as adjuvants for an FMD recombinant protein (C-terminal half of the VP1 protein) vaccine; it was found that a combination of recombinant protein in ISCOMs with Montanide ISA-206 could achieve early protective titres and longer lasting immunity in guinea pigs; it remains to be seen whether FMD vaccines formulated in ISCOMs will be as successful in target species. Also, this technology is rather expensive.

The adjuvant effects of synthetic cytidine-phosphate-guanosine (CpG) oligodeoxynucleotides (ODNs) on FMD vaccines were also evaluated. The studies demonstrated that a combination of CpG ODNs with ISA-206 could facilitate a recombinant FMDV vaccine A7 (containing multiple B- and T-cell epitopes) to induce a vigorous and long-lasting specific antibody response in mice and cattle. However, CpG combined with an FMD inactivated vaccine did not promote protection (Cao, supra;). (Alves et al., 2009, Pigs. Clin. Vaccine Immunol., vol. 16, p. 1151-1157).

In view of the above, there is a continuing need for FMD vaccine adjuvants suitable to induce the desired immunological enhancement such as strong and long-lasting humoral and cellular immunity, while complying with general requirements relating to safety, stability and ease of use, in order to overcome the drawbacks of the currently available adjuvants in FMDV emulsion vaccines.

However, as vaccine development is a highly empirical process, and different types of response are required to protect against FMDV, there is little justification for concluding that one particular adjuvant will be significantly more useful than other available alternatives (Cao, supra, p. 1381, right column).

In view of the above, there is a continued need in the field of veterinary vaccinology to provide affordable methods and materials that allow the formulation of safe, stable and effective adjuvated vaccines that comprise bacterial-, parasitic-, and/or viral antigens.

It is therefore an object of the present invention to overcome a disadvantage in the prior art, and to accommodate to this need in the field by providing stable adjuvated emulsions of bacterial-, parasitic-, and/or viral antigens that can be used as effective vaccines.

### SUMMARY OF THE INVENTION

The present invention provides an adjuvant composition comprising an emulsion of water, a tocopherol or a pharmaceutically acceptable ester thereof, and a polyethoxyethylene cetostearyl ether as an emulsifier. The adjuvant composition can advantageously be used for formulating an emulsion vaccine.

The invention further provides a vaccine composition comprising said adjuvant composition and an antigen.

The invention also provides a method for the manufacture of a vaccine composition comprising the claimed adjuvant composition and an antigen, comprising the steps of:
a) preparing an aqueous phase comprising an antigen, and
b) admixing said aqueous phase with the adjuvant composition as claimed.

Further, the invention provides the claimed vaccine composition for use in a method of protecting a human or animal target against infection and/or disease caused by a pathogen.

Details and preferences of an adjuvant composition according to the invention and a vaccine composition formulated therewith will be described herein below.

### LEGEND TO THE FIGURES

FIG. 1:

Microscopic views of sample IP.1 (Eumulgin in the waterphase) at t=0 (FIG. 1A), after 10 days at 37° C. (FIG. 1i), and after 3 weeks at 37° C. (FIG. 1C).

Microscopic views of sample IP.2 (Eumulgin in the oily phase) at t=0 (FIG. 1D), after 10 days at 37° C. (FIG. 1E), and after 3 weeks at 37° C. (FIG. 1F).

Microscopic views of sample IP.3 (Polysorbate 80 in the waterphase) at t=0 (FIG. 1G), after 10 days at 37° C. (FIG. 1H), and after 3 weeks at 37° C. (FIG. 1I).

FIG. 2:

Vaccination of cattle with an Asial/Shamir SVEA-E/FMDV vaccine composition, high virus-neutralizing titres were found (panel 2 A), which also provided full protection against an FMDV challenge infection (panel 2 B). Details are in Example 8.

FIG. 3:

Duration of immunity study in cattle using SVEA-E-adjuvanted FMD vaccine composition with O/TUR/5/2009 antigen, given as prime and boost, and compared to a corresponding Montanide ISA206-adjuvanted vaccine composition.

FIG. 4:

Duration of immunity study in pigs using SVEA-E-adjuvanted FMD vaccine composition with O/TUR/5/2009 antigen, given as single immunisation, and as compared to a corresponding Montanide ISA206-adjuvanted vaccine composition.

FIG. 5:

Duration of immunity study in goats using SVEA-E-adjuvanted FMD vaccine composition with O/TUR/5/2009 antigen, given as single immunisation, and as compared to a corresponding Montanide ISA206-adjuvanted vaccine composition.

FIG. 6:

Test of the stability of FMDV capsid at 4° C. over time, when comprised in a vaccine composition formulated with SVEA-E adjuvant. Details are in Example 8.

FIG. 7:

Graphic representation of the Mastersizer results for each of the samples IP.1, IP.2, and IP.3 (top to bottom) of Example 4, measured after 10 days at 37° C.

### DETAILED DESCRIPTION

During research into oil-adjuvanted emulsion vaccines with multiple bacterial antigens, the inventors observed occasional failures in the stability of the emulsions. The emulsions were found to break (i.e. showed loss of dispersion, and increase of phase-separation) already after some weeks in stability tests, rendering the vaccine ineffective. Evident possible causes such as poor batch quality of the used mineral oil, or of the used emulsifiers (Polysorbate 80 [Tween] and Sorbitan mono-oleate [Span]), were quickly ruled out, leaving the problem unsolved. Without any indication on where to start, began a long investigation into the observed breaking of the emulsions: both for the factor that was affected, and for the factor causing that effect.

Surprisingly it was found that the object can be met, and consequently one or more disadvantages of the prior art can be overcome, by an adjuvant composition comprising an emulsion of water, a tocopherol or a pharmaceutically acceptable ester thereof, and a polyethoxyethylene cetostearyl ether. This was found to solve the occurrence of emulsion-instability, even in complex mixtures, and even when using relatively crude preparations of antigens.

This finding is hugely beneficial for the development of vaccines based on emulsions of water and oil. Especially as it overcomes a need to modify the quality or the composition of the non-live bacterial-, parasitic-, and/or viral antigens used in such an emulsion vaccine, thus allowing the use of relatively impure non-live bacterial, parasitic, and/or viral antigen preparations in emulsion vaccines. In fact, the antigen can now even be used at a lesser purity than before and/or at a higher concentration in the vaccine, while there is no need to establish the level of impurity: antigens of all purity-levels can now be formulated into a stable emulsion vaccine of the invention, using nothing but routine skills.

It is not known exactly how an adjuvant composition comprising particularly an emulsion of water, a tocopherol or a pharmaceutically acceptable ester thereof, and a polyethoxyethylene cetostearyl ether as an emulsifier, provides stability to emulsions of oil and water that contain non-live antigens. Although the inventors do not want to be bound by any theory or model that might explain these findings, they speculate that the sorbitan-based emulsifiers used in the prior art, in particular Polysorbate, were being degraded over time, leading to a loss of emulsifying capacity, and the subsequent breaking of the emulsion. They also speculate that the factors degrading the prior art emulsifiers were enzymes that were present in the crude preparation of the non-live antigens. Of the various enzymes present: proteases, esterases, carbohydrases and nucleases, the esterases may have been the cause for degrading the emulsifiers, considering that an increase in free fatty acid levels could be observed to correlate with a reduction of the concentration of the prior art emulsifiers, in emulsions that showed breaking.

Such esterase enzymes are produced by many of the known bacterial and parasitic genera and in vivo may serve as virulence factor, and/or to mobilise nutritional components. All major families of bacteria, both Gram-positive and Gram-negative, are involved, and both benign or pathogenic strains, such as: Bacillus, Staphylococcus, Pseudomonas, Salmonella, and many more. Consequently such enzymes can be present, in principle, in any preparation of non-live bacterial antigens. A similar consideration applies to crude preparations of non-live parasitic antigens.

In view of the above it is particularly surprising that out of a huge variety of possible emulsifiers that can replace the sorbitan-based emulsifiers used in the prior art such as Polysorbate, the specific adjuvant composition according to the invention comprising a tocopherol or a pharmaceutically acceptable ester thereof, and a polyethoxyethylene cetostearyl ether, can be used as an effective emulsifier which was found not to be sensitive to the effect of esterases.

This was not at all obvious from any disclosure in the prior art, as an emulsion vaccine is a complex formulation with a multitude of biological- and chemical compounds, for which it is very difficult to determine which factor is affected, and which component of the composition is the cause, upon the observation of instability of the emulsion.

There is a large number of potential ingredients in a typical emulsion vaccine with non-live bacterial-, parasitic, and/or viral antigens, of which any one or more can have a negative effect on one of the other components, for example:
components of the culture medium used for growing the bacterial culture: e.g. animal serum, broth or extract of animal tissues, peptones or hydrolysates of animal or vegetable origin, lipids, sugars, amino acids, vitamins and minerals, antifoam, antifungals, etc.
(traces of) inactivating agent: e.g. thiomersal, benzalkonium chloride, formaldehyde, betapropiolactone, or detergent, etc.
stabilisers: dextrane, glycerol, gelatine, amino acids, buffers, etc.
preservatives: thiomersal, phenoxyethanol, formaldehyde, antibiotics (e.g. gentamycin),
components of the micro-organism: fragments of the cell wall, the internal cellular organelles, the nucleic acid; external bacterial organelles such as: pili, fimbriae, or flagella;
bacterial proteins, lipids, and carbohydrates, such as: toxins, lipopolysaccharides, various enzymes, etc.
one or more adjuvant(s), emulsifier(s), and their trace elements or impurities.

Further the stability of the emulsion may be affected by one or more effects from physical factors in the preparation, purification, formulation, emulsification, filling, transport and storage of the vaccines.

Finally, the inventors had no clue towards the selection of a suitable alternative for a Polysorbate emulsifier, especially as that replacing emulsifier had to provide the emulsion with the required stability in the context of the various other ingredients of an emulsion vaccine such as the oily adjuvants and the antigens employed.

Thus, in a first aspect, the invention relates to an adjuvant composition comprising an emulsion of water, a tocopherol or a pharmaceutically acceptable ester thereof, and a polyethoxyethylene cetostearyl ether.

In said composition, the tocopherol or the pharmaceutically acceptable ester thereof acts as an oily adjuvant, and the polyethoxyethylene cetostearyl ether acts as an emulsifier.

The water used for the adjuvant composition according to the invention, is preferably water of high purity, and is of pharmaceutical quality grade and suitable for parenteral injection; such quality water is typically sterile, and essentially free from pyrogens, and is for example: (multiply) distilled water, reverse osmosis water, or water for injection (WFI).

An “adjuvant” is a substance that increases or modulates the immune response, e.g. to a vaccine.

In particular, an adjuvant provides an immune-stimulation for an antigen, particularly a non-live antigen, which would otherwise not be sufficiently immunogenic. This will trigger different routes of the immune system, however the basic mechanisms of this process are not well understood.

An “emulsion” is a mixture of at least two immiscible liquids, whereby one is dispersed in another. Typically the droplets of the dispersed phase are very small, with diameters of a few micrometres or less.

An emulsifier for the invention is a molecule with amphiphilic properties, having both a hydrophobic- and a hydrophilic side. Many emulsifiers are known in the art with their various properties. Most are readily available commercially, and in different degrees of purity.

As used herein, the term “polyethoxyethylene cetostearyl ether” refers to a class of hydrophilic, non-ionic emulsifiers that are used in the manufacturing of various O/W emulsions. This term is used interchangeably in this application with the term “polyoxyethylene cetostearyl ether” Polyoxyethylene cetostearyl ether is an ether of cetostearyl alcohol. Cetostearyl alcohol, cetearyl alcohol or cetylstearyl alcohol is a mixture of fatty alcohols, consisting predominantly of cetyl (16 C) and stearyl alcohols (18 C) and is classified as a fatty alcohol. In the present application, the term “polyethoxyethylene cetostearyl ether” has the same meaning as “a mixture comprising a polyethoxyethylene cetyl ether and a polyethoxyethylene stearyl ether”.

Particular examples include polyethoxyethylene 12 cetostearyl ether (INCI: Ceteareth-12, CAS number: 68439-49-6, Ph. Eur.: Macrogol cetostearyl ether 12), polyethoxyethylene 20 cetostearyl ether (Ceteareth-20, CAS number: 68439-49-6, Macrogol cetostearyl ether 20) and polyethoxyethylene 30 cetostearyl ether (Ceteareth-30, CAS number: 68439-49-6).

Polyethoxyethylene 12 cetostearyl ether has an HLB (Hydrophilic-Lipophilic Balance) number of 14 and is a mix of the following components:
polyethoxyethylene 12 cetyl ether (cetyl=C16):

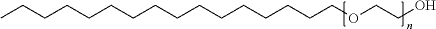

with n=12
polyethoxyethylene 12 stearyl ether (stearyl=C18):

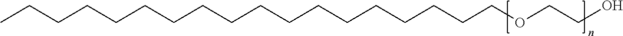

with n=12

Polyethoxyethylene 12 cetostearyl ether is commercially available under a variety of commercial names, e.g. as Eumulgin® B1, Kolliphor® CS 12, Volpo® CS 12, Cremophor® A25, Simulsolt™, or Brij CS12®.

Polyethoxyethylene 12 cetostearyl ether finds application in antiperspirants and deodorants, sun care (after-sun, self-tanning, sun protection), body-, colour- and face care products. It is also used in baby care and cleansing, face cleansing, hair colouring and conditioning formulations. It is further used as a mild, non-ionic emulsifier for pharmaceutical oil-in-water emulsions; delivering good sensory properties during product application.

Polyethoxyethylene 12 cetostearyl ether is a component of the adjuvant known as AF03, which comprises: Squalene (NB: thus not squalane), Eumulgin® B1 (polyethoxyethylene 12 cetostearyl ether), Montane™ 80 (sorbitan oleate), Mannitol, and phosphate buffered saline.

“Tocopherol”, as used herein, refers to a class of organic chemical compounds having vitamin E activity. Tocopherol includes α-Tocopherol (CAS number: 10191-41-0), β-Tocopherol (CAS number: 148-03-8), γ-Tocopherol (CAS number: 54-28-4), and δ-Tocopherol (CAS number: 119-13-1).

Pharmaceutically acceptable esters of tocopherol (vitamin E) particularly include alpha-tocopheryl-acetate which is also termed tocopheryl acetate or vitamin E-acetate having CAS number 58-95-7. Alpha-tocopheryl-acetate can be derived from vegetable materials such as seeds, nuts, fruits or leaves, or from fatty meats, but may also be produced synthetically. Thus, included in the definition of vitamin E-acetate are natural, synthetic or semi-synthetic forms, or mixtures thereof. Vitamin E-acetate is commercially available, in different degrees of purity.

An alpha-tocopheryl-acetate for the adjuvant composition according to the invention is preferably DL-alpha-tocopherol-acetate, which is the racemate of the chemical with CAS number: 7695-91-2.

The term “comprising” (as well as variations such as “comprise”, “comprises”, and “comprised”) as used herein, refer(s) to all elements, and in any possible combination conceivable for the invention, that are covered by or included in the text section, paragraph, claim, etc., in which this term is used, even if such elements or combinations are not explicitly recited; and does not refer to the exclusion of any of such element(s) or combinations. Consequently, any such text section, paragraph, claim, etc., can also relate to one or more embodiment(s) wherein the term “comprises” (or its variations) is replaced by terms such as “consist of”, “consisting of”, or “consist essentially of”.

The adjuvant composition may further comprise a pharmaceutically acceptable carrier.

The adjuvant composition may be identical to the vaccine composition as described herein, but lacking an antigen.

A “pharmaceutically acceptable carrier” for the invention is an aqueous liquid of a high grade of purity and preferably sterile, for example: water, a physiological salt solution, or a phosphate buffered saline solution. The carrier can comprise further additives, such as stabilisers or preservatives.

In a preferred embodiment of the adjuvant composition according to the invention, the polyethoxyethylene cetostearyl ether is selected from polyethoxyethylene 12 cetostearyl ether, polyethoxyethylene 20 cetostearyl ether, and polyethoxyethylene 30 cetostearyl ether. Even more preferably, the polyethoxyethylene cetostearyl ether is polyethoxyethylene 12 cetostearyl ether.

In another embodiment of the adjuvant composition according to the invention, the pharmaceutically acceptable ester of tocopherol is an alpha-tocopheryl acetate.

In a preferred embodiment, the adjuvant composition according to the invention comprises polyethoxyethylene 12 cetostearyl ether and alpha-tocopheryl acetate.

In a preferred embodiment, the adjuvant composition according to the invention is free of ester surfactants, such as esters of fatty acids, particularly of esters formed from sugar alcohols or their derivatives and fatty acids such as sorbitan or mannide esters. Such esters are commonly used as surfactants in oil-based adjuvant compositions. Sorbitan esters are also known as polysorbates, Tweens or Spans. Particular examples of esters include polyoxyethylene sorbitan mono-oleate (Polysorbate 80 or Tween® 80), sorbitan monostearate (Span 60), sorbitan tristearate (Span 65), sorbitan monolaurate (Span 20). Other examples include esters of octadecanoic acid and anhydromannitol (contained in Montanide ISA-206).

Preferably also the vaccine composition according to the invention is free of esters of fatty acids.

In another embodiment, the adjuvant composition according to the invention further comprises squalane.

“Squalane” is a well-known compound, and preferably refers to the chemical compound with CAS number 111-01-3. Some alternate names are: hydrogenated shark liver oil, hexamethyltetracosane, or perhydrosqualene. This is not to be confused with squalene (CAS nr. 111-02-4) which is a poly-unsaturated C30 oil and is metaboliseable as a compound of the cholesterol pathway. However, squalane is the fully hydrogenated form of squalene and is therefore not prone to oxidation. Thus, while squalane is a non-mineral oil, and is transported from the injection site, it is non-metaboliseable.

Originally the precursor to squalane was obtained from shark livers, but over environmental concerns this has shifted to other natural sources, such as olive oil, or to chemical synthesis. Therefore included in the definition of squalane are natural, synthetic or semi-synthetic forms, or mixtures thereof. Squalane is commercially available in a variety of purities, for example: from vegetable source, from Worlee (Squalane, vegetable), or Croda (Pripure Squalane); or synthetic, e.g. from Kuraray (Squalane-PE). For the invention, a high purity of the squalane is preferred: preferably over 75% purity, more preferably over 80, 90, or even over 95% purity, in that order of preference.

In an embodiment, the adjuvant composition according to the invention further comprises a mineral oil.

Examples of mineral oil suitable for use in the adjuvant composition include, for example, Bayol™ or Markol™, Montanide™ or a light (or white) liquid paraffin oil, such as Marcol® 52 (Exxon Mobile) or Drakeol® 6VR (Penreco) or Klearol® (Sonneborn).

In an embodiment, the adjuvant composition according to the invention further comprises a non-mineral oil.

Non-mineral oils can be of synthetic, animal or vegetable origin. For use in the adjuvant composition of the invention the non-mineral oils are preferably biodegradable (metabolisable) and biocompatible. Examples of synthetic oils suitable for use in the adjuvant composition include, for example, a Shell Ondina® oil, e.g. Shell Ondina X420. Examples of animal oils suitable for use in the adjuvant composition include, for example, fish oil. Examples of vegetable oils suitable for use in the adjuvant composition include oils derived from nuts, seeds and grains, such as peanut oil, soybean oil, coconut oil, and olive oil; jojoba oil; safflower oil, cottonseed oil, sunflower seed oil, sesame seed oil; corn oil, or the oil of other cereal grains such as wheat, oats, rye, rice, teff, and triticale.

In a particularly preferred embodiment the adjuvant composition according to the invention comprises polyethoxyethylene 12 cetostearyl ether, alpha-tocopheryl acetate, and squalane.

In another particularly preferred embodiment the adjuvant composition according to the invention comprises polyethoxyethylene 12 cetostearyl ether, alpha-tocopheryl acetate, and a non-mineral oil. Preferably, the non-mineral oil is a synthetic oil, such as a Shell Ondina X GTL-based medicinal white oil.

In another particularly preferred embodiment the adjuvant composition according to the invention comprises polyethoxyethylene 12 cetostearyl ether, alpha-tocopheryl acetate, and a mineral oil. Preferably, the mineral oil is a light liquid paraffin oil.

In an embodiment, the adjuvant composition according to the invention is an oil-in-water emulsion.

An “oil-in-water emulsion” is a well-known composition, comprising an outer aqueous phase, which contains an internal dispersed oily phase; for the invention, the dispersed phase is formed by the droplets of the oily adjuvant (tocopherol or a pharmaceutically acceptable ester thereof).

By the selection of the appropriate kind and concentration of emulsifier(s), such emulsions can be formed. Procedures and equipment for the preparation of an oil-in-water emulsion or water-in-oil emulsion for use as a vaccine are well-known in the art, and are described in handbooks known to a skilled person.

An “oily phase” (or “oily phase”) is a liquid based on an oil. An “oil” is used herein in its common meaning and refers to a nonpolar chemical substance that is hydrophobic and lipophilic, with a high hydro-carbon content. An oil can be of mineral origin, or of non-mineral such as of synthetic, animal or vegetable origin. Some non-mineral oils are metabolisable.

The oily phase may contain excipients such as an emulsifier. For the invention, the oil-phase is the dispersed phase, as is typical for an O/W emulsion. When formulated into a vaccine, the oil-phase serves as adjuvant. Much used mineral oil adjuvant in veterinary vaccines is a light liquid paraffin oil, such as Marcol® (Exxon Mobile) or Drakeol® (Penreco). Common non-mineral oil adjuvants are squalene and squalane (shark liver oil), and tocopherol (Vitamin E).

An “aqueous phase” is a liquid based on water. The aqueous phase may contain e.g. a buffer or saline, and one or more excipients such as an emulsifier or a stabiliser.

The size of the particles of the dispersed phase of the inventive adjuvant composition is preferably quite small. When the average diameter of the particles of the dispersed phase is below about 1 micrometre, such emulsions are commonly called “submicron emulsions”.

In an embodiment of the O/W emulsion of the adjuvant composition according to the invention, the emulsion is a submicron emulsion.

Equipment to measure particle sizes of 1 micrometre or less is generally available, for example by laser diffraction measurement. Typically particle size is expressed in nanometres (nm), and as an average particle size, also known as median diameter, expressed as the D50 of a cumulative particle size distribution.

For the invention, particle size is expressed in nm of D50, as determined using a Mastersizer™ (Malvern Instruments). Particle size measurements can be made in the (concentrated) oily emulsion constituting the adjuvant composition or the vaccine composition; the particle refractive index of the oily phase for the invention is 1.48. The Malvern Mastersizer size analysis report presents D50 as D(0.50).

There are many ways available to produce such submicron emulsions, typically by the use of a high-energy emulsification process, for example using: high-pressure homogenisers, rotor-stator devices, blenders, ultrasonic waves, microporous membranes, or microchannelling devices.

Preferred process for high-energy emulsification for preparing the adjuvant composition according to the invention, is the use of a high-pressure homogeniser, preferably a Microfluidiser™ (Microfluidics). Typically 1-3 passages at a pressure of between 500-1500 bar (i.e. 7000-22000 psi) will be sufficient.

Emulsions prepared in this way typically have dispersed phase particles with a D50 of 500 nm or less, and have a narrow size distribution. Such an emulsion is called a nano emulsion.

Typically emulsions with such very finely sized particles of the dispersed phase, are prepared in several consecutive steps. In this way, an initial relatively coarse oily emulsion is prepared by low-energy mixing, which is followed by one or more subsequent high-energy treatments to achieve further reduction of particle size.

Next, the ‘microfluidised’ oily emulsion, comprising the adjuvant(s) and emulsifier in water is then combined with the aqueous phase with or without the antigens, to prepare the adjuvant composition, respectively the vaccine composition according to the invention.

Therefore, in an embodiment of the submicron oil-in-water emulsion of the adjuvant composition or the vaccine composition according to the invention, the oil-droplets, i.e. (emulsion) particles, have a D50 of 500 nm or less; preferably D50 is 250 nm or less. More preferred: D50 is 150 nm or less. Particularly preferably, the emulsion particles have a particle size D50 of about 80 to about 200 nm, more preferably of about 40 to about 100 nm.

For reasons of product consistency and—quality, not only the median particle diameter, but also the spread in particle size, also known as the size distribution, can advantageously be monitored and controlled. The size distribution of the oil-droplets in the submicron oil-in-water emulsion of the adjuvant composition or the vaccine composition according to the invention is preferably relatively narrow. An indicator of particle size distribution is the D90 of a cumulative particle size distribution.

Therefore, in an embodiment of the submicron oil-in-water emulsion of the adjuvant composition or the vaccine composition according to the invention, the oil-droplets have a D90 below 900 nm, more preferred D90 is below 500 nm, 400 nm, or even below 300 nm, in that order of preference. Most preferred: D90 is equal to or smaller than about 250 nm.

One of the advantages of the emulsion having such small particle size, and—distribution, is that this can then be sterilised by filtration, without significant loss of material. This because typical sterilisation filters have a pore size of about 0.2 micrometres. Such filter sterilisation overcomes the need for other methods of sterilisation that may be damaging to the quality of the components of the oily emulsion, such as by: heating, chemicals, or irradiation.

Thus, in one embodiment the adjuvant composition or the vaccine composition according to the invention comprises emulsion particles having a particle size D50 of about 80 to about 200 nm, preferably of about 40 to about 100 nm.

In the adjuvant composition according to the invention the polyethoxyethylene cetostearyl ether emulsifier is present in an amount of about 1 to about 9% by weight based on the weight of the adjuvant composition (w/w). Preferably, the polyethoxyethylene cetostearyl ether is present in an amount of about 2 to about 7% w/w of the adjuvant composition.

In a particularly preferred embodiment the polyethoxyethylene cetostearyl ether is present in an amount of about 6 to about 7% w/w of the adjuvant composition.

In another particularly preferred embodiment the polyethoxyethylene cetostearyl ether is present in an amount of about 2 to about 4% w/w of the adjuvant composition.

For the invention “about” indicates that a number can vary between +25% around its indicated value. Preferably “about” means±20% around its value, more preferably “about” means±15, 12, 10, 8, 6, 5, 4, 3, 2% around its value, or even “about” means±1% around its value, in that order of preference.

In the adjuvant composition according to the invention a tocopherol or a pharmaceutically acceptable ester thereof is present in an amount of about 1 to about 20% by weight based on the weight of the adjuvant composition. Preferably, the tocopherol or a pharmaceutically acceptable ester thereof is present in an amount of about 6 to about 16% w/w of the adjuvant composition.

In a particularly preferred embodiment the tocopherol or a pharmaceutically acceptable ester thereof is present in an amount of about 14 to about 16% w/w of the adjuvant composition. In another particularly preferred embodiment the tocopherol or a pharmaceutically acceptable ester thereof is present in an amount of about 2 to about 8% w/w of the adjuvant composition. Most preferred: the tocopherol or a pharmaceutically acceptable ester thereof, particularly alpha-tocopheryl acetate, is present in an amount of about 15% w/w of the adjuvant composition.

In the adjuvant composition according to the invention squalane is present in an amount of about 5 to about 20% by weight based on the weight of the adjuvant composition. Preferably, squalane is present in an amount of about 6 to about 15% w/w of the adjuvant composition. In a particularly preferred embodiment squalane is present in an amount of about 13 to about 14% w/w of the adjuvant composition.

In the adjuvant composition according to the invention the mineral oil is present in an amount of about 5 to about 40% by weight based on the weight of the adjuvant composition. In a particularly preferred embodiment the mineral oil is present in an amount of about 6 to about 7% w/w of the adjuvant composition.

In another particularly preferred embodiment the mineral oil is present in an amount of about 30 to about 40% w/v of the adjuvant composition, preferably about 35% w/v of the adjuvant composition.

In the adjuvant composition according to the invention the non-mineral oil is present in an amount of about 5 to about 40% by weight based on the weight of the adjuvant composition. In a particularly preferred embodiment the non-mineral oil is present in an amount of about 6 to about 7% w/w of the adjuvant composition.

In another particularly preferred embodiment the non-mineral oil is present in an amount of about 30 to about 40% w/v of the adjuvant composition, preferably about 35% w/v of the adjuvant composition.

The adjuvant composition according to the invention can be prepared as a concentrate. Said concentrate can be, for example, a 2× w/v concentrate which means that the concentrate represents 50% of the final volume of the vaccine. Analogously, if the concentrate is, for example, a 4× w/w concentrate, this means that the concentrate represents 25% of the weight of the final vaccine, and so forth.

In a particularly preferred embodiment the adjuvant composition comprises about 5 to about 7% w/w, preferably about 6% w/w of polyethoxyethylene 12 cetostearyl ether, and about 14 to about 16% w/w, preferably about 15% w/w of alpha-tocopheryl acetate. Herein, this adjuvant composition is referred to as Fortasol-E. Fortasol-E is a 2× w/v concentrate of the final vaccine which means that the concentrate represents 50% of the volume of the final vaccine. The Fortasol-E formulation is a nano emulsion.

In a particularly preferred embodiment the adjuvant composition according to the invention comprises about 6 to about 7% w/w of polyethoxyethylene 12 cetostearyl ether, about 15 to about 16% w/w of alpha-tocopheryl acetate and about 13 to about 14% w/w of squalane.

Herein, this adjuvant composition is referred to as SVEA-E. SVEA-E is a 4× w/v concentrate, which is comprised as 25% of the volume of the final vaccine. The SVEA-E adjuvant composition is a nano emulsion.

In another preferred embodiment the adjuvant composition according to the invention comprises about 3 to about 4% w/w of polyethoxyethylene 12 cetostearyl ether, about 7 to about 8% w/w of alpha-tocopheryl acetate and about 6 to about 7% w/w of a non-mineral oil. Herein, this adjuvant composition is referred to as EV0420. EV0420 is a 2× w/v concentrate which is comprised as 50% of the volume of the final vaccine. The EV0420 adjuvant composition is a nano emulsion.

Surprisingly, a vaccine composition according to the invention comprising the SVEA-E adjuvant composition and further comprising Orf2 antigen of PCV2 and crude bacterin antigen of Mhyo, was found to be stable even after 27 months at 4° C. A similar vaccine composition with SVEA-E and comprising FMDV empty capsids as the antigen was found to be stable up to 8 months at 4° C.

In an embodiment, the adjuvant composition according to the invention further comprises silica.

Preferably the silica is “fumed silica”; more preferably the fumed silica is present in an amount of about 0.3% w/w to about 0.5% w/w, more preferably in an amount of about 0.4% w/w of the adjuvant composition.

“Fumed silica” as used herein refers to silicium dioxide with an average particle size of about 6 to about 13 nm. Fumed silica is commercially available under the tradename Aerosil 380 (average particle size of 7 nm). Pharmaceutical quality grade fumed silica is commercially available as Aerosil 300 (average particle size of 7 nm, which has a smaller surface area compared to Aerosil 380) or Aerosil 200 (average particle size of 12 nm).

In a further preferred embodiment the adjuvant composition according to the invention comprises about 6 to about 7% w/w of polyethoxyethylene 12 cetostearyl ether, about 15 to about 17% w/w of alpha-tocopheryl acetate, about 13 to about 14% w/w of squalane and further about 0.3 to 0.5% w/w of fumed silica. Herein, this adjuvant composition is referred to as SVEA-E double plus. SVEA-E double plus is a 2× w/w concentrate, which is comprised as 50% of the weight of the final vaccine. The SVEA-E double plus adjuvant composition is a nano emulsion.

In a further preferred embodiment the adjuvant composition according to the invention comprises only one third of the amount of emulsifier of the SVEA-E composition, i.e. about 2 to about 2.3% w/w of polyethoxyethylene 12 cetostearyl ether. Herein, this adjuvant composition is referred to as microSVEA-E. The microSVEA-E adjuvant composition has emulsion particles in the micrometre range and, therefore, represents a microemulsion.

Analogously, microSVEA-E double plus comprises only one third of the amount of emulsifier of the SVEA-E double plus composition, i.e. about 2 to about 2.3% w/w of polyethoxyethylene 12 cetostearyl ether. Herein, this adjuvant composition is referred to as microSVEA-E double plus. The microSVEA-E double plus adjuvant composition has emulsion particles in the micrometre range and, therefore, represents a microemulsion.

In a particularly preferred embodiment the adjuvant composition according to the invention comprises about 2 to about 3% w/w polyethoxyethylene 12 cetostearyl ether, about 2 to about 3% w/w alpha-tocopheryl acetate and about 30 to about 40% w/v; preferably about 35% of a non-mineral oil. Herein, this adjuvant composition is referred to as Xsolve2.0. The Xsolve2.0 adjuvant composition is a nano emulsion.

The adjuvant composition according to the invention can be used for formulating an emulsion vaccine, e.g. a vaccine composition according to the invention.

For the invention, ‘formulation’ regards the preparation of the vaccine composition according to the invention, by the admixing of an aqueous phase comprising one or more antigens as described herein, and the adjuvant composition according to the invention.

The adjuvant composition according to the invention can be used for formulating a non-live vaccine, e.g. a vaccine composition according to the invention comprising a non-live antigen as described herein.

In an embodiment, the adjuvant composition according to the invention does not contain (i.e. is free from) an emulsifier that is susceptible to degradation resulting from a crude bacterial- or parasitic antigen as defined for the invention; preferably susceptible to degradation by an esterase or lipase as defined herein. More preferably, the emulsion does not contain a sorbate-based emulsifier, or even: does not contain a Polysorbate; even more preferably: does not contain a Polysorbate and a Sorbitan mono-oleate.

In a further aspect, the invention relates to a vaccine composition comprising the adjuvant composition according to the invention and an antigen.

An “antigen” is a substance that is capable of inducing an immunological reaction in a target human or animal, possibly with the help of an immunostimulating compound such as an adjuvant. Antigens can be prepared synthetically or can be derived from a biological source, for example they can be a microorganism (replicative or not), or can be a part thereof, e.g. a protein, lipid, carbohydrate, or nucleic acid, or combinations thereof, e.g.: a peptidoglycan, a lipoglycan, a lipopeptide, or a lipopolysaccharide, etc.

For the invention, non-live (non-replicating) antigen relates to molecules such as proteins, carbohydrates, lipids or nucleic acids, or are complex combinations thereof, more or less pure. When prepared from a microorganism, non-live antigen can refer to a killed (i.e. non-replicative) microorganism, or can be a part thereof such as an extract, fraction, homogenate, or sonicate. Also a non-live antigen can be a nucleic acid based, or recombinant product, such as an expression vector or an expressed protein, or the product of an in vitro expression system. All these are well-known in the art.

“Live” antigen refers to live (i.e. replicative) bacteria, parasites, or viruses that are suitable for use as a vaccine component, i.e. having a reduced level of pathogenicity, also known as being attenuated, or modified live.

As used herein, “attenuated” is defined as causing a lower level of lesions, and/or having a reduced rate of infection, or of replication. All, as compared to unmodified or ‘wildtype’ bacteria, parasites, or viruses.

Attenuation of a micro-organism can be obtained in vitro, for instance by passageing through experimental animals or in cell-culture and selection, or via recombinant DNA technology, all well known in the art.

While it is biologically incorrect to refer to a virus as being “live”, that is the common way to refer to a virus that is replicative and not inactivated. Consequently, for the invention the term “live” as relating to a virus refers to a virus that is capable of replication under appropriate conditions, e.g. in suitable host cells or animals.

The vaccine composition according to the invention comprises an antigen of a bacterium, a parasite, and/or a virus. The antigen is preferably a non-live antigen. Optionally the vaccine composition can comprise one or more further antigens, e.g. from a bacterium, parasite, virus, fungus, ectoparasite, etc. The further antigen can be live or non-live.

Therefore, in a preferred embodiment of the vaccine composition according to the invention, the vaccine composition comprises one or more antigens selected from a non-live antigen of a bacterium, of a parasite, and of a virus. Preferably, the vaccine composition contains multiple (two or more) antigens, more preferably two or more bacterial antigens.

A “bacterial antigen” is an antigen that is derived, based on, or obtained from a bacterium. For the invention, a bacterium is a prokaryotic microorganism that is currently classified in the taxonomic super kingdom of Bacteria.

A “parasite antigen” is derived, based on, or obtained from a parasite. For the invention, a parasite is a eucaryotic microorganism; for example as currently classified in the clade Apicomplexa, or the phylum Nematoda.

Ectoparasites are e.g. flies, flees, mites, or ticks.

Fungi are for example Aspergillus.

A “viral antigen” is an antigen that is derived, based on, or obtained from a virus. The antigen included in the vaccine composition according to the invention is derived from a micro-organism that is pathogenic to the target species which is to be protected against infection and/or disease by the vaccine.

Examples of micro-organisms pathogenic to porcine animals are:
Viruses: porcine circovirus type 2, porcine parvovirus, African swine fever virus, swine influenza virus, porcine respiratory coronavirus, porcine cytomegalovirus, porcine rotavirus, porcine poxvirus, porcine reproductive and respiratory syndrome virus (PRRSV), pseudorabies virus, classical swine fever virus, swine influenza virus, foot-and-mouth disease virus, porcine epidemic diarrhoea virus, transmissible gastro-enteritis virus, and vesicular stomatitis virus.
Bacteria: Mycoplasma, Lawsonia, Escherichia, Brachyspira, Streptococcus, Salmonella, Clostridium, Actinobacillus, Pasteurella, Haemophilus, Erysipelothrix, Leptospira, Burkholderia, Enterococcus, Mycobacterium, and Bordetella.
Parasites: Toxoplasma, Isospora, and Trichinella.

Examples of micro-organisms that are pathogenic to ruminants are:
For cattle: Neospora, Dictyocaulus, Cryptosporidium, Ostertagia, bovine rotavirus, bovine viral diarrhoea virus, bovine coronavirus, infectious bovine rhinotracheitis virus (bovine herpes virus 1), bovine paramyxo virus, bovine parainfluenza virus, bovine respiratory syncytial virus, rabies virus, bluetongue virus, Escherichia, Salmonella, Staphylococcus, Streptococcus Mycobacterium, Brucella, Clostridium, Pasteurella, Yersinia Mannheimia, Haemophilus, Leptospira, Bacillus, Moraxella, Francisella, Histophilus, Trueperella, Fusobacterium. Actinomyces, Coxiella, Campylobacter, Erysipelothrix, Listeria, Burkholderia, Nocardia, Mycoplasma, Bacteroides, and Chlamydia.

For sheep and goats: Toxoplasma gondii, peste des petit ruminant virus, bluetongue virus, Schmallenberg virus, Mycobacterium, Brucella, Clostridia, Coxiella, Escherichia, Chlamydia, Clostridia, Pasteurella, and Mannheimia.

For cervines: Epizootic haemorrhagic disease virus, bluetongue virus, papilloma virus, Borrelia burgdorferi, Mycobacterium bovis, and Trueperella pyogenes.

Particularly advantageously, the vaccine composition according to the invention can comprise a crude preparation of a bacterial or parasitic antigen. Because of the emulsion-stabilizing effect provided by the components of the adjuvant composition according to the invention, no elaborate and expensive purification of bacterial and/or parasitic antigens is required.

The expression “crude” refers to an antigen from a bacterium, parasite or virus, as used herein, which was prepared without much (or any) further purification after they were harvested from an in vitro culture, and inactivated and/or lysed or disrupted. Such crude antigen preparations still contain undefined or unintended impurities which can have an effect on the stability of a vaccine based on an emulsion of oil and water.

The antigen to be comprised in the vaccine composition according to the invention, is typically contained in a liquid, such as an aqueous buffer.

As described, the advantageous effect of the present invention applies particularly to the use of such vaccine composition for the agricultural sector.

In an embodiment of the vaccine composition according to the invention, the antigen is a non-live bacterial antigen; preferably the non-live bacterial antigen is an inactivated whole bacterial culture (i.e. a bacterin), or is part of such culture.

Preferably the part of the inactivated whole bacterial culture is selected from: a pellet, supernatant, concentrate, dialysate, extract, sonicate, lysate, and fraction of such a culture. What constitutes a “bacterial culture” or “a part thereof” is well-known to a skilled person, and is described in handbooks and manuals such as “Veterinary vaccinology” (supra). For the invention the inactivated bacterial culture is used either as a whole, i.e. as the complete content of a particular culture vessel, or as a part thereof.

Methods and materials to prepare such an inactivated culture or to prepare such a part thereof are generally known and available at any scale. For example: inactivation of bacteria can be performed using chemical or physical means; physical means are e.g. heating, irradiation, or very high pressure; chemical means are e.g. incubation with merthiolate, formalin, diethylamine, binary ethylenamine, beta propiolactone, benzalkonium chloride or glutaraldehyde.

A supernatant or a pellet can be prepared by centrifugation.

A concentrate or a dialysate can be prepared e.g. by a method of cross-flow filtration.

An extract can be made for example by washing or incubation with a solvent or a detergent solution; the solvent can be a liquid or a gas, the liquid can e.g. be aqueous such as water or a buffer; an organic solvent such as an alcohol, acetone, or ether; or can be a supercritical liquid, etc. The extract is the part that is removed with the solvent, and is often retrieved from that solvent in a subsequent process.

A sonicate can be prepared using a sonification device, for example a flow-through sonification cell.

A lysate can be prepared by physical or (bio-)chemical means, e.g. using a French press, or using an enzymatic treatment.

A fraction is a part of a whole that is purified from the rest, for example a filtrate or a precipitate, whereby the fraction is the retentate.

Most used in bacterial vaccines for agricultural use, is a non-live bacterial antigen which comprises inactivated bacterial cells. Commonly such an antigen preparation of killed bacterial cells is called a bacterin.

The inactivated bacterial cells can be in any form, and can be intact or can be damaged. The inactivated bacterial cells can be at any level of purity, for example can be with the bacterial culture medium in which they were fermented, or can be without the culture medium, for example resulting from sedimentation, centrifugation, or concentration.

Therefore in an embodiment of the vaccine composition according to the invention, the non-live bacterial antigen comprises inactivated bacterial cells.

The bacteria from which the non-live antigen is prepared can be any bacterium of human medical- and/or of veterinary relevance, for example any bacterium that is a (potential) pathogen, either as primary- or as secondary (opportunistic) pathogen.

By way of example, suitable bacterial antigens include Actinobacillus pleuropneumoniae, Mycoplasma hyopneumoniae (Mhyo), Lawsonia intracellularis, Erysipelothrix rhusiopathiae, and Leptospira, or a part of any of those, alone or in any combination.

Evidently, combinations of non-live bacterial antigen from two or more bacteria can also be prepared and used. Also, combinations with antigens from viral- or parasitic pathogens are desired.

The bacterial antigen is preferably a crude antigen as described herein; more preferably the crude bacterial antigen is a bacterin from Mhyo or from L. intracellularis.

For the invention a viral antigen is preferably derived from an in vitro culture of virus and host cells; the virus may be obtained from the culture supernatant, from the pellet, or may be comprised in the host cells. A wide variety of pathogenic viruses are known to be relevant to veterinary medicine.

Exemplary viral antigens include PCV2, PRRSV and FMDV, or a part of any of those, alone or in any combination.

An antigen for the invention may also be comprised in the vaccine composition according to the invention by way of a recombinant vector; such a vector may be a nucleic acid or a replicon particle (RP); the nucleic acid is preferably a eukaryotic expression plasmid or an RNA molecule; the RP is preferably an Alphavirus RP.

In an embodiment of the vaccine composition according to the invention the antigen comprises a viral antigen. It is preferred that the viral antigen comprises FMDV, preferably non-live antigen of FMDV, e.g. recombinantly expressed virus-like particles of FMDV. The FMDV antigen is particularly preferably a recombinantly expressed empty capsid of FMDV.

In an embodiment of the vaccine composition according to the invention, the non-live antigen is selected from one or more of porcine circovirus type 2, Mycoplasma hyopneumoniae, and foot-and-mouth disease virus. Preferably one or more further non-live antigen may be comprised, selected from PRRSV, or L. intracellularis.

A “vaccine” is a well-known composition with a medical effect, and comprises an immunologically active component, and a pharmaceutically acceptable carrier. As ‘carrier’ for the invention functions the aqueous phase, or the adjuvant composition according to the invention itself. The ‘immunologically active component’ for the invention is the antigen from a bacterium, parasite, or virus, or is a combination of antigens from one or more of those micro-organisms.

The vaccine composition according to the invention stimulates the immune system of a target human or animal, and induces a protective immunological response. The response may originate from the targets' innate- and/or from the acquired immune system, and may be of the cellular—and/or of the humoral type.

A vaccine provides protection “against infection and/or disease” by reducing in a vaccinated target the severity of an infection, for example by reducing the number of pathogens, or shortening the duration of the pathogen's replication in the target, and reducing the number, the intensity, or the severity of lesions caused by infection with a pathogen. Also, or consequentially, a vaccine is effective in reducing or ameliorating the (clinical) symptoms of disease that may be caused by such infection or replication, or by the target's response to that infection or replication. A reference for such diseases and clinical signs is: “The Merck veterinary manual” (supra). Such a vaccine is colloquially referred to as a: vaccine ‘against’ the particular pathogen, e.g. as an ‘Mhyo vaccine’, or as an ‘FMDV vaccine’.

In order to be immunologically effective, a vaccine needs to contain a sufficient amount of the antigen. How much that is, is either already known from related vaccines, or can readily be determined e.g. by monitoring the immunological response following vaccination and (in the case of an animal target) a challenge infection, e.g. by monitoring the targets' signs of disease, clinical scores, or by re-isolation of the pathogen, and comparing these results to a vaccination-challenge response seen in mock-vaccinated animals.

The amount of the bacterial-, parasitic-, or viral antigen for the vaccine composition according to the invention, can be expressed in different ways, depending on the type of the antigen employed. For example the antigen dose can be expressed as a number of bacterial cells, parasites, or virions, counted before they were inactivated. Alternatively the antigen can be quantified by a serologic- or bio-chemical test such as an ELISA or an AlphaLisa™, and expressed in relative units, compared to an appropriate reference standard. All these are well known in the art.

The vaccine composition according to the invention can be used as a prophylactic-, metaphylactic-, or therapeutic treatment.

The vaccine composition according to the invention can serve as an effective priming vaccination, which can later be followed and amplified by a booster vaccination, with the same or with a different vaccine.

The vaccine composition according to the invention can additionally comprise other compounds, such as an additional antigen or micro-organism, a cytokine, or an immunostimulatory nucleic acid comprising an unmethylated CpG, etc. Alternatively, the vaccine composition according to the invention, may itself be added to a vaccine.

As described, the vaccine composition according to the invention is of particular relevance in the field of animal husbandry.

Consequently, for the different groups of animal targets, the non-live bacterial antigen for the invention is derived from a bacterium a bacterial family such as:
For vaccination of ruminants the bacterium is selected from one of: Pasteurella, Escherichia, Salmonella, Yersinia, Staphylococcus, Streptococcus, Mycobacterium, Moraxella, Bacillus, Brucella, Clostridia, Mannheimia, Haemophilus, Francisella, Fusobacterium, Histophilus, Fusobacterium, Trueperella (Arcanobacterium), Actinomyces, Clostridia, Coxiella, Campylobacter, Erysipelothrix, Leptospira, Listeria, Burkholderia, Nocardia, Mycoplasma, Bacteroides, and Chlamydia.
For vaccination of porcines the bacterium is selected from one of: Mycoplasma, Lawsonia, Escherichia, Brachyspira, Streptococcus, Salmonella, Clostridia, Actinobacillus, Pasteurella, Haemophilus, Erysipelothrix, Leptospira, Burkholderia, Enterococcus, Mycobacterium, and Bordetella.

In an even more preferred embodiment of the vaccine composition according to the invention, the non-live antigen is from porcine circovirus type 2, Mycoplasma hyopneumoniae, and/or foot-and-mouth disease virus.

It is well within reach of the skilled person to further optimise a vaccine composition according to the invention. Generally this involves the fine-tuning of the efficacy of the vaccine to further improve its provided immune-protection. This can be done by adapting the dose, volume, adjuvant or antigen content of the vaccine, or by application via a different route, method, or regime. All these are within the scope of the invention.

In the vaccine composition according to the invention the emulsifier is present in an amount of about 1 to about 5% by weight based on the volume of the vaccine composition (w/v). Preferably, the emulsifier is present in an amount of about 1 to about 4% w/v of the vaccine composition. In a particularly preferred embodiment the emulsifier is present in an amount of about 1 to about 2% w/v of the vaccine composition. In another particularly preferred embodiment the emulsifier is present in an amount of about 2 to about 3% w/v of the vaccine composition. In another particularly preferred embodiment the emulsifier is present in an amount of about 3 to about 4% w/v of the vaccine composition. In another particularly preferred embodiment the emulsifier is present in an amount of about 0.3 to about 1.2% w/v of the vaccine composition.

In the vaccine composition according to the invention a tocopherol or a pharmaceutically acceptable ester thereof is present in an amount of about 2 to about 9% by weight based on the volume of the vaccine composition. Preferably, the tocopherol or a pharmaceutically acceptable ester thereof is present in an amount of about 3 to about 8% w/v of the vaccine composition. In a particularly preferred embodiment the tocopherol or a pharmaceutically acceptable ester thereof is present in an amount of about 2 to about 3% w/v of the vaccine composition. In another particularly preferred embodiment the tocopherol or a pharmaceutically acceptable ester thereof is present in an amount of about 3 to about 4% w/v of the vaccine composition. In another particularly preferred embodiment the tocopherol or a pharmaceutically acceptable ester thereof is present in an amount of about 7 to about 8% w/v of the vaccine composition.

In the vaccine composition according to the invention squalane is present in an amount of about 3 to about 7% by weight based on the volume of the vaccine composition. Preferably, squalane is present in an amount of about 3 to about 4% w/v of the vaccine composition. Also preferably, squalane is present in an amount of about 6 to about 7% w/v of the vaccine composition.

In the vaccine composition according to the invention the mineral oil is present in an amount of about 3 to about 40% by weight based on the volume of the vaccine composition. In a preferred embodiment the mineral oil is present in an amount of about 3 to about 4% w/v of the vaccine composition. In another preferred embodiment the mineral oil is present in an amount of about 30 to about 40% w/v of the vaccine composition, preferably about 35% w/v of the vaccine composition.

In the vaccine composition according to the invention the non-mineral oil is present in an amount of about 3 to about 40% by weight based on the volume of the vaccine composition. In a preferred embodiment the noon-mineral oil is present in an amount of about 3 to about 4% w/v of the vaccine composition. In another preferred embodiment the non-mineral oil is present in an amount of about 30 to about 40% w/v of the vaccine composition, preferably about 35% w/v of the vaccine composition.

In a particularly preferred embodiment the vaccine composition is based on the Fortasol-E adjuvant composition and comprises about 5 to about 7% w/v, preferably about 6% w/v of polyethoxyethylene 12 cetostearyl ether, and about 14 to about 16% w/v, preferably about 15% w/v of alpha-tocopheryl acetate. The density of a vaccine composition based on the Fortasol-E adjuvant composition is about 0.9 to about 1.1 g/ml, preferably about 0.9 to about 1.0 g/ml.

In a particularly preferred embodiment the vaccine composition is based on the SVEA-E adjuvant composition and comprises about 1 to about 2% w/v of polyethoxyethylene 12 cetostearyl ether, about 3 to about 4% w/v of alpha-tocopheryl acetate and about 3 to about 4% w/v of squalane. The density of a vaccine composition based on the SVEA-E adjuvant composition is about 0.9 to about 1.1 g/ml, preferably about 0.9 to about 1.0 g/ml.

A vaccine based on the SVEA-E double plus adjuvant composition is particularly suitable for intradermal administration, e.g. by means of the IDAL® device, particularly for intradermal administration to swine.

In a particularly preferred embodiment the vaccine composition is based on the EV0420 adjuvant composition and comprises about 1 to about 2% w/v of polyethoxyethylene 12 cetostearyl ether, about 3 to about 4% w/v of alpha-tocopheryl acetate and about 3 to about 4% w/v of squalane. The density of a vaccine composition based on the EV0420 adjuvant composition is about 0.9 to about 1.1 g/ml, preferably about 0.9 to about 1.0 g/ml.

In a particularly preferred embodiment the vaccine composition is based on the SVEA-E double plus adjuvant composition and comprises about 3 to about 4% w/v of polyethoxyethylene 12 cetostearyl ether, about 7 to about 8% w/v of alpha-tocopheryl acetate, about 6 to about 7% w/v of squalane, and about 0.1 to 0.3% w/v of fumed silica. The density of a vaccine composition based on the SVEA-E double plus adjuvant composition is about 0.9 to about 1.1 g/ml, preferably about 0.9 to about 1.0 g/ml.

In a particularly preferred embodiment the vaccine composition is based on the microSVEA-E adjuvant composition and comprises about 0.3 to about 0.7% w/v of polyethoxyethylene 12 cetostearyl ether, about 3 to about 4% w/v of alpha-tocopheryl acetate and about 3 to about 4% w/v of squalane. The density of a vaccine composition based on the microSVEA-E adjuvant composition is about 0.9 to about 1.1 g/ml, preferably about 0.9 to about 1.0 g/ml.

In a particularly preferred embodiment the vaccine composition is based on the microSVEA-E double plus adjuvant composition and comprises about 0.8 to about 1.2% w/v of polyethoxyethylene 12 cetostearyl ether, about 7 to about 8% w/v of alpha-tocopheryl acetate, about 6 to about 7% w/v of squalane, and about 0.1 to 0.3% w/v of fumed silica. The density of a vaccine composition based on the microSVEA-E double plus adjuvant composition is about 0.9 to about 1.1 g/ml, preferably about 0.9 to about 1.0 g/ml.

Vaccine compositions according to the invention based on microSVEA-E or on microSVEA-E double plus as the adjuvant composition according to the invention, are particularly suitable for intramuscular administration.

As such vaccine compositions based on microSVEA-E or on microSVEA-E double plus, are both microemulsions, these thus have dispersed particles in the emulsion in the micrometre range. However, and surprisingly, these vaccine compositions did not exhibit any problems with stability such as breaking of the emulsion. Therefore, using an adjuvant composition that is a microSVEA-E or a microSVEA-E double microemulsion allows to obtain vaccine compositions having larger emulsion particles in the micrometre range, which nevertheless have good safety and no issues with stability. This can be used advantageously in relation to protection against certain pathogens and disease, as vaccines with a larger dispersed particle size can be expected to induce a different (i.e. less Th1-directed) type of immune response than do vaccines with nano emulsions

In a particularly preferred embodiment the vaccine composition is based on the Xsolve2.0 adjuvant composition and comprises about 2 to about 3% w/w polyethoxyethylene 12 cetostearyl ether, about 2 to about 3% w/w alpha-tocopheryl acetate and about 30 to about 40% w/v; preferably about 35% of a non-mineral oil. The density of a vaccine composition based on the Xsolve2.0 adjuvant composition is about 0.9 to about 1.1 g/ml, preferably about 0.9 to about 1.0 g/ml.

In an embodiment, the vaccine composition has a high payload of antigen(s). The total antigen concentration is preferably at least 20% v/v, preferably at least 25% v/v, more preferably at least 30% v/v of the vaccine composition.

The vaccine composition according to the invention is preferably an emulsion vaccine.

The vaccine composition according to the invention was found to be very effective, safe and stable, when prepared as an oil-in-water emulsion.

Therefore, in a preferred embodiment the vaccine composition according to the invention is an oil-in-water emulsion vaccine.

If the vaccine composition according to the invention is an oil-in-water emulsion vaccine the aqueous antigen can be combined with the adjuvant composition using low shear mixing techniques, as adjuvant composition itself has already been emulsified, potentially involving high shear mixing.

It is preferred that the outer aqueous phase comprises the antigen in a pharmaceutically acceptable carrier; and the oily phase comprises alpha-tocopheryl acetate and optionally squalane as oily adjuvant(s).

The O/W emulsion according to the invention may itself be used for the formulation of a further emulsion, such as a W/0/W or O/W/O emulsion. This may require the use of an additional emulsifier. Selection and optimisation of such conditions are within the capabilities of the skilled person.

As detailed above, it was surprisingly found that the adjuvant composition of the invention is suitable for formulating a vaccine comprising crude antigens, i.e. little purified antigens derived from, for example, an inactivated bacterial or—parasite culture, or from extracts or fractions of such a culture.

Therefore, in an embodiment of the vaccine composition according to the invention, the antigen comprises a crude antigen.

Since no elaborate and expensive purification of bacterial- and/or parasitic antigens is required, the vaccine composition according to the invention is cost effective. Furthermore, reduced batch-to-batch variability was observed, along with suitability of the inventive composition for large-scale production.

Further, because of the properties of the adjuvant composition according to the invention, it is particularly useful for combination with antigens, particularly non-live bacterial—or—parasitic antigens, that contain an enzyme with esterase activity; such antigens can deteriorate the emulsifier used in prior art emulsions of an oily phase and an aqueous phase, causing the emulsion to break.

Thus, in an embodiment of the vaccine composition according to the invention, the antigen comprises an esterase, preferably a lipase.

As used herein, the term “esterase” refers to enzymes having esterase activity, specifically to esterases and lipases. Such enzymes are characterised in the IUBMB (International Union of Biochemistry and Molecular Biology) enzyme classification system under EC 3.1.1.1 or EC 3.1.1.3 respectively.

Tests to determine whether an antigen “contains an esterase” for the invention, refer to tests for esterase activity which exist in a great variety of forms and types of assays, and are available commercially.

A straightforward detection of esterase activity is by spectrophotometric assay, by detecting a yellow coloration from the release of nitrophenol from a p-nitrophenyl-ester by an active esterase. The paper by De Yan et al. (2013, Biotechn. & Appl. Biochem., vol. 60, p. 343-347) lists several variants of this type of assay in its references no. 2-16. All these are variations on the basic assay as described by De Caro et al. (1986, Eur. J. of Biochem., vol. 158, p. 601-607).

For the invention, to establish whether an antigen contains an esterase, a detection test is performed according to De Caro et al. (supra), using as substrate: p-nitrophenyl-butyrate. The skilled person is perfectly capable of setting up and performing such a test.

Any level of esterase activity in the antigen can be harmful to the stability of prior art emulsions, especially upon long-term storage. Therefore based on appropriate positive and negative reference standards, the presence of any detectable level of esterase activity qualifies an antigen as ‘contains an esterase’ according to the invention.

The adjuvant composition according to the present invention, and particularly a vaccine composition comprising said adjuvant composition in combination with an antigen, surprisingly exhibit several further benefits.

In particular, while it is not straightforward to find an adjuvant that works well across species, the adjuvant composition and the vaccine composition, both according to the invention, are successfully used for porcines and for ruminants; preferably used for pigs, cattle, sheep and goats.

Further, the adjuvant composition and vaccine composition derived therefrom is comparatively easy to administer since its density is low as compared to prior art adjuvant compositions. Preferably, the density of the adjuvant is from about 0.9 to about 1.1 g/ml, preferably from about 0.9 to about 1.0 g/ml.

Additionally, the adjuvant composition and the vaccine composition derived therefrom is safe, as no serious adverse events were observed.

As a favourable feature of an O/W emulsion, it is possible to determine the antigen content in the vaccine composition according to the invention, without breaking the vaccine composition directly in an ELISA plate.

The vaccine composition comprising high antigen amounts, such as in multivalent- or combination vaccines, is considerably more stable as compared to prior art compositions.

The adjuvant composition and vaccine composition derived therefrom can tolerate higher temperatures as compared to prior art compositions, e.g. Montanide ISA206 which phase-reverses above 32° C.

Very surprisingly, the vaccine composition according to the invention provides quick onset of immunity (OOI).

Further surprisingly, the vaccine composition according to the invention provides for a longer duration of immunity (DOI).

The vaccine composition according to the invention can be prepared using well-known methods and materials. The details of these procedures will be dependent on the type of the emulsion to be prepared. For example, as an O/W emulsion, the emulsification of the oily- and aqueous phase (without antigen) can be done separately (e.g. to constitute the adjuvant composition according to the invention), before the subsequent admixing of the antigen to prepare the emulsion vaccine according to the invention.

Occasionally it may be required to apply some heating during preparation of the adjuvant composition, for example to 70-90° C., to get the emulsifier completely dissolved. When required, further emulsifiers can be comprised in the oil and/or in the aqueous phase. Commonly, the aqueous- and the oily phases can be emulsified using suitable equipment such as by ultrasonic-, or rotor-stator type mixing.

The inventors further discovered that the polyethoxyethylene cetostearyl ether of the adjuvant composition according to the invention dissolved surprisingly well into the oily phase. This was unexpected for an emulsifier of high HLB number (e.g. Polyethoxyethylene 12 cetostearyl ether has an HLB of 14), especially as Polysorbate 80 having almost the same HLB number (15) does not dissolve well in the oil phase.

This now allows to employ methods of emulsification that require lower shear and less power input, which leads to various savings and operational advantages.

Alternatively, for making an O/W type emulsion, where the antigen is typically not present during the initial emulsification of oily phase, emulsifier, and water, the use of high intensity emulsification of water and oily phase is still a further option, for example by using microfluidisation.

Therefore in a further aspect the invention relates to a method for the manufacture of a vaccine composition according to the invention, the method comprising the steps of:
a) preparing an aqueous phase comprising an antigen, and
b) admixing said aqueous phase with the adjuvant composition according to the invention.

For the method for the manufacture of a vaccine according to the invention, each of: the aqueous phase, the oily phase, the adjuvant composition, and the antigen, are as defined herein.

In an alternative embodiment, an aqueous phase may be prepared including an antigen and the emulsifier (i.e. a polyethoxyethylene cetostearyl ether). Separately an oily phase including a tocopherol or a pharmaceutically acceptable ester thereof, and optionally squalane, a mineral oil, and/or a non-mineral oil, is prepared prior to admixing both phases in order to obtain the vaccine composition of the invention.

Preferably the method for the manufacture according to the invention is performed in a way that allows a medical use of the vaccine composition. Commonly this regards the use of equipment and excipients that are pharmaceutically acceptable, and complying with quality regulations such as good manufacturing practice standards. All these are well known to a skilled person, and are prescribed in Governmental regulations such as the Pharmacopoeia, and in handbooks such as: Remington and Pastoret (both supra). Typically such manufacture is done aseptically.

As described, the vaccine composition according to the invention is particularly advantageous when applied as a vaccine against bacterial-, parasitic-, and/or viral disease, all as described herein.

Therefore, in a further aspect the invention relates to the vaccine composition according to the invention for use in a method of protecting a human- or animal target against infection and/or disease caused by a pathogen.

The selection of the target for the method of protecting according to the invention is mainly determined by the host range of the pathogen to be protected against: for humans, for animals, or for both. Alternatively, a pathogen can be pathogenic to humans but not to an animal carrying the pathogen. In that case it may still make sense to apply animal vaccination, in order to prevent zoonotic infection and food-borne illness of humans that could consume an infected animal product such as e.g. meat or milk.

The age, weight, sex, immunological status, and other parameters of the target to be vaccinated are not critical, although it is clearly favourable to vaccinate healthy, uninfected targets, and to vaccinate as early as possible.

An “animal” for the invention is any animal of veterinary relevance, e.g. bovine, porcine, caprine, ovine, cervine, canine, feline, equine, avian, fish, or shrimp.

In an embodiment of the vaccine composition for use in a method of protecting according to the invention, the animal target is a swine or a ruminant.

The term “swine” refers to animals of the family Suidae, and preferably to animals of the genus Sus, which are also referred to as porcines. Examples are: a wild or a domestic pig, hog, wild boar, babirusa, or warthog. This also includes swine indicated by an arbitrary name, for example referring to their sex or age such as: sow, queen, boar, barrow, hog, gilt, weaner, or piglet. Further the term swine refers to porcine animals of any type such as of breeding- or fattening type, and to parental lines of any of these types.

The term “ruminant” refers to large hoofed herbivorous grazing or browsing mammals that are able to acquire nutrients from plant-based food by fermenting it in a specialized stomach prior to digestion, principally through microbial actions. Ruminating mammals include cattle, all domesticated and wild bovines, goats, sheep, giraffes, deer, gazelles, and antelopes.

In a preferred embodiment of the vaccine composition for use in a method of protecting an animal target, wherein the target is a ruminant, the ruminant is selected from agricultural livestock, particularly cattle, bovines, sheep, and goats.

In a particularly preferred embodiment of the vaccine composition for use in a method of protecting an animal target, wherein the target is a swine, the swine is a sow or a young swine. A ‘young swine’ for the invention is a swine of less than 6 months old, preferably less than 5, 4, 3, 2, or even less than 1 month old, in this order of preference.

In an embodiment of the vaccine composition for use in a method of protecting according to the invention, the pathogen is selected from a bacterium, a parasite, and a virus, all as described herein.

Preferably the pathogen is selected from one or more of porcine circovirus type 2, Mycoplasma hyopneumoniae, and foot-and-mouth-disease virus.

In an embodiment of the vaccine composition for use in a method of protecting according to the invention, the vaccine composition is administered by intradermal or intramuscular route.

In a further aspect the invention relates to the vaccine composition according to the invention for use in the vaccination of a human or animal target against infection and/or disease caused by a pathogen.

In an embodiment of the vaccine composition for use in the vaccination according to the invention, the animal target is a ruminant or a swine, and/or the pathogen is a bacterium, parasite, or virus, all as described herein.

As the skilled person will be well aware, the vaccine composition according to the invention can be applied “for use in a method of protecting” or “for use in the vaccination” in different ways. For example, the vaccine composition itself can be applied as a vaccine. Alternatively the vaccine composition can be used as ingredient in further processing for example into a W/O/W or O/W/O emulsion, which can then be applied as a vaccine. Also, the use as a vaccine may involve admixing or including certain further ingredients, for example stabilisers or preservatives. Preservatives are e.g. thiomersal, phenoxyethanol, formalin, antibiotics (e.g. gentamycin). Stabilisers are e.g. dextrane, glycerol, gelatine, amino acids, or buffers. Depending on the type of the emulsion, the further ingredients may be added during or after the manufacture of the vaccine composition according to the invention.

In a further aspect the invention relates to the use of the adjuvant composition according to the invention for the manufacture of a vaccine composition for protecting a human or animal target against infection and/or disease caused by a pathogen.

In an embodiment of the use according to the invention, the animal target is a ruminant or a swine, and/or the pathogen is a bacterium, parasite, or virus, all as described herein.

The vaccine composition according to the invention needs to be administered to a human or animal target, in order to achieve its beneficial immunogenic effect.

Therefore, in a further aspect the invention relates to a method for the vaccination of a human or animal target against infection and/or disease caused by a pathogen, the method comprising the administration to said target of the vaccine composition according to the invention.

In an embodiment of the method for the vaccination according to the invention, the animal target is a ruminant or a swine, and/or the pathogen is a bacterium, parasite, or virus, all as described herein.

The “administration” of the vaccine composition according to the invention to a human or animal target can be performed using any feasible method and route. Typically the optimal route and method of administration will be determined by the type of the vaccine applied, and by the characteristics of the target and of the disease that it is intended to protect against. Different techniques of administration can be applied. For example as an O/W emulsion, the vaccine composition according to the invention is aqueous in character, and can therefore be administered by enteral or mucosal route, i.e. via eye drop, nose drop, oral, enteric, oro-nasal drop, spray. Other possibility is via a method of mass administration, such as via drinking water, coarse spray, atomisation, on-feed, etc. A skilled person is perfectly capable of selecting and optimising such route- and method of administration.

Preferred way of administration for a method of vaccination according to the invention is by parenteral route.

“Parenteral” refers to administration through the skin, for example by intramuscular, intraperitoneal, intradermal, submucosal, or subcutaneous route.

In an embodiment of the method for the vaccination according to the invention, the vaccine composition is administered by intradermal- or intramuscular route.

The volume of a dose of the vaccine composition according to the invention, e.g. when administered by parenteral route, is a volume that is acceptable for the target human or animal, and can for instance be between about 0.1 and about 10 ml. Preferably one dose is a volume between 0.1 and 5 ml, more preferably one dose is between 0.2 and 3 ml.

When administered by intramuscular route, the volume of one dose is preferably between about 0.5 and about 3 ml, more preferably between 1 and 2 ml.

When administered by intradermal route, the volume of one dose is preferably between about 0.1 and about 0.5 ml, more preferably is about 0.2 ml.

The method, timing, and volume of the administration of a vaccine composition according to the invention is preferably integrated into existing vaccination schedules of other vaccines that the target human or animal may require, in order to reduce stress to the target and to reduce labour costs. These other vaccines can be administered in a simultaneous, concurrent or sequential fashion, in a manner compatible with their registered use.

The adjuvant composition according to the invention can be marketed as a stand-alone product. This allows it to be used by qualified persons for preparing an O/W emulsion vaccine with a preferred antigen shortly before administration. Such on-the-spot preparation is also called field-side mixing, and allows flexibility in the selection and the combination of the antigens that are admixed. However a vaccine composition prepared in such a way does not have the guaranteed properties of quantity, quality, and sterility that are provided with a ready-mixed vaccine composition that was prepared in the pharmaceutical factory of a registered manufacturer.

An alternative is the provision of the adjuvant composition according to the invention in a kit of parts. Such a kit is typically a package comprising 2 or more containers, the content of which can be mixed to prepare the vaccine composition according to the invention shortly before administration. For the invention, the kit of parts can comprise one container having the adjuvant composition according to the invention, and one or more further containers comprising one or more antigens. The one or more further antigens can be in liquid form.

Alternatively, and advantageously, the one or more further antigens can be in a dried-, or in a freeze-dried form. This employs the advantageous properties of the adjuvant composition of the invention as an O/W emulsion: as that behaves as an aqueous composition, it can be used as a diluent to reconstitute a dried- or a freeze-dried antigen. The freeze-dried antigen can be a live—or a killed micro-organism, for example can be a freeze-dried preparation of live PRRSV, or can be a freeze-dried preparation of non-live L. intracellularis.

Therefore in a further aspect the invention relates to a kit of parts, the kit comprising at least two containers, one container comprising the adjuvant composition according to the invention, and one container comprising an antigen.

In an embodiment of the kit of parts according to the invention, the antigen is a freeze-dried live PRRSV, or is a freeze-dried preparation of non-live L. intracellularis.

The kit of parts according to the invention may comprise a box with said two or more containers, and may comprise instructions for use. Said instructions may for example be written on the box that contains the constituents of the kit; may be present on a leaflet in- or on- or with that box; or may be viewable on, or downloadable from, an internet website from the manufacturer or the distributor of the kit, etc.

The invention will now be further described by the following, non-limiting, examples.

### EXAMPLES

### Example 1: Fortasol-E

Fortasol-E is a 2× w/v concentrate. As a reference, a similar composition having Polysorbate 80 (PS80) instead of Eumulgin B1 as the emulsifier was prepared.

### Composition Per 100 g:

Oily phase

| | | |
|---|---|---|
| DL-α-tocopherol acetate | 15.00 | g |
| Eumulgin B1 | 6.00 | g |

Water phase

| | | |
|---|---|---|
| WFI | 79.00 | g |

### Preparation:

Heat oil and water phase to 75-85° C.
Add oily phase slowly to the water phase while stirring with visible vortex
After addition continue stirring for 1 minute
Filtration through 0.2 μm filter
Mix 1:1 with Mhyo antigen for stability at 37° C.

The Mhyo antigen used was a crude antigen preparation of a bacterin of Mhyo grown in a culture using pig blood in the medium.

### Results

Oily phase with Eumulgin B1 was clear, the oily phase with Polysorbate 80 was turbid
Both adjuvant compositions can be easy filtrated through a 0.2 μm Nylon filter
The adjuvant composition with Eumulgin showed some slight clustering of the visible particles
Macroscopically both adjuvant compositions appear as a nano emulsion, with a blue glow

### Mastersizer Results in μm:

| Adjuvant | | 0.2 μm | d | d | d | d | d |
|---|---|---|---|---|---|---|---|
| composition | Emulsifier | filtration | (0.10) | (0.50) | (0.90) | (0.99) | (1.00) |
| Concentrate | Eumulgin | No | 0.07 | 0.13 | 0.23 | 0.66 | 1.18 |
| | B1 | | | | | | |
| | Polysorbate | No | 0.08 | 0.13 | 0.22 | 2.10 | 8.45 |
| | 80 | | | | | | |
| | Eumulgin | Yes | 0.08 | 0.14 | 0.23 | 0.68 | 1.03 |
| | B1 | | | | | | |
| | Polysorbate | Yes | 0.08 | 0.13 | 0.22 | 0.74 | 1.18 |
| | 80 | | | | | | |
| Concentrate + | Eumulgin | Yes: t = 0 | 0.08 | 0.14 | 0.23 | 0.69 | 1.03 |
| Mhyo 1:1 | B1 | 55 days | 0.08 | 0.14 | 0.23 | 0.67 | 1.18 |
| | | at 37° C. | | | | | |
| | Polysorbate | Yes: t = 0 | 0.07 | 0.12 | 0.21 | 0.81 | 1.37 |
| | 80 | 55 days | 0.20 | 0.33 | 0.71 | 123.74 | 236.38 |
| | | 37° C. | | | | | |

### Conclusion

It was possible to prepare a Fortasol 2× concentrate with Eumulgin B 1 instead of Polysorbate 80, as the emulsifier.
Fortasol-E was stable in a 1:1 ratio with Mhyo antigen, even after 55 days at 37° C., the reference with Polysorbate 80 showed breaking and increase in particle size

### Example 2: SVEA-E

SVEA-E is a 4× w/v concentrate. The concentrate formed 25% w/v of the final vaccine.

The final vaccine contained the following components and strengths:
Eumulgin B1 (0.941 gr/ml): 1.62% w/v
Squalane (d=0.81 gr/ml): 3.375% w/v
Vitamin E Acetate (0.953 gr/ml): 3.97% w/v
Density of final vaccine=0.9892 gr/ml

The O/W emulsion was prepared as a 4× w/v concentrate. WFI was used as water phase. High pressure homogenization was used.

### Composition 4× w/v Concentrate:

### Per 100 g (104.36 ml)

Oily phase

| | | |
|---|---|---|
| Eumulgin B1 | 6.48 | g |
| Squalane-PE (Kuraray) | 13.50 | g |
| DI-alpha tocopherol acetate | 15.88 | g |

(corrected for purity)

Water phase

| | | |
|---|---|---|
| WFI | ad 100.00 | g |

### Preparation:

### Oily Phase

1. Weigh the amount of Squalane.
2. Add the amount of Vitamin E Acetate and homogenize until it is a clear mixture.
3. Add the amount of Eumulgin B1 and homogenize at 70° C. A clear mixture appeared.
4. At a temperature of 70° C. sterilize this mixture by filtration using a 0.2 μm membrane filter in a filter house provided with a double jacket to keep the temperature at 70° C. during filtration.
5. Cool down to 55-65° C.

### Water Phase

6. Use sterilized (autoclaved) Water For Injection. Heat it up to 55-65° C.

### Final Product

7. Prepare premix by adding oily phase to the water phase while mixing with a magnetic stirrer or an overhead stirrer at 900 rpm
8. Continue mixing for 5 minutes
9. Treat the premix, 3 passages through a (sterilized) microfluidizer at 800 bar (temp 55-75° C.)

The SVEA-E 4× concentrate microscopically appeared as an O/W solubilisate with some droplets between 1 and 5 μm.

### Example 3: SVEA-E Double Plus

SVEA-E Double Plus is a 2× w/w concentrate.

Densities of the concentrate and of the final vaccine have been measured:
Density concentrate=0.965 g/ml
Density final vaccine=0.986 g/ml

The final vaccine contained the following components and strengths:
Squalane (d=0.81 g/ml): 6.77% w/v
Vitamin E Acetate (0.953 g/ml): 7.97% w/v
Macrogol cetastearyl ether NS (0.941 g/ml): 3.25% w/v
Aerosil 300 pharma: 0.20% w/v

### Composition:

### Per 100 g Concentrate (=50% w/w of the Final Vaccine)

| | | |
|---|---|---|
| Squalane | 13.74 | g |
| Vitamin E acetate (corrected for purity) | 16.17 | g |
| Macrogol Cetostearyl Ether 12 NS | 6.59 | g |
| Aerosil 300 pharma | 0.41 | g |
| Water for injection | ad 100.00 | g |

### Preparation:

1. Weigh WFI, Aerosil 300 and Eumulgin B1 (=water phase), homogenize at 40-50° C. until the Eumulgin B1 dissolves. Sterilize 20 minutes at 121° C. while stirring
2. Weigh Squalane and Vit E acetate (=oily phase), homogenize until a clear composition appears. Sterilize by 0.2 μm filtration at 70° C.
3. Add oily phase to the water phase while stirring at 55-65° C. with a magnetic stirrer, visible vortex
4. Continue stirring for 5 minutes at 55-65° C.
5. Treat premix 3 passages at 800 bars with microfluidizer at 55-75° C.

### Example 4: Mhyo Antigen in SVEA-E Double Plus

The SVEA-E double plus composition comprised in the final vaccine: 3.25% w/v of polyethoxyethylene 12 cetostearyl ether; 7.97% w/v of alpha-tocopheryl acetate; 6.77% w/v of squalane; and 0.20% w/v of fumed silica.

The SVEA double plus reference composition comprised in the final vaccine: 3.24% w/v of Polysorbate 80; 7.94% w/v of alpha-tocopheryl acetate; 6.75% w/v of squalane; and 0.20% w/v of fumed silica.

### Compositions:

| IP | Adjuvant | Emulsifier | Mhyo antigen |
|---|---|---|---|
| 1 | SVEA-E Double Plus | Eumulgin B1 in water | Whole water |
| | | phase | phase |
| 2 | | Eumulgin B1 oily phase | |
| 3 | SVEA Double Plus | Polysorbate 80 in water | |
| | (reference) | phase | |

### Results

Appearance after 10 days at 37° C.

| | |
|---|---|
| IP.1: | No breaking |
| IP.2: | No Breaking |
| IP.3: | Broken |

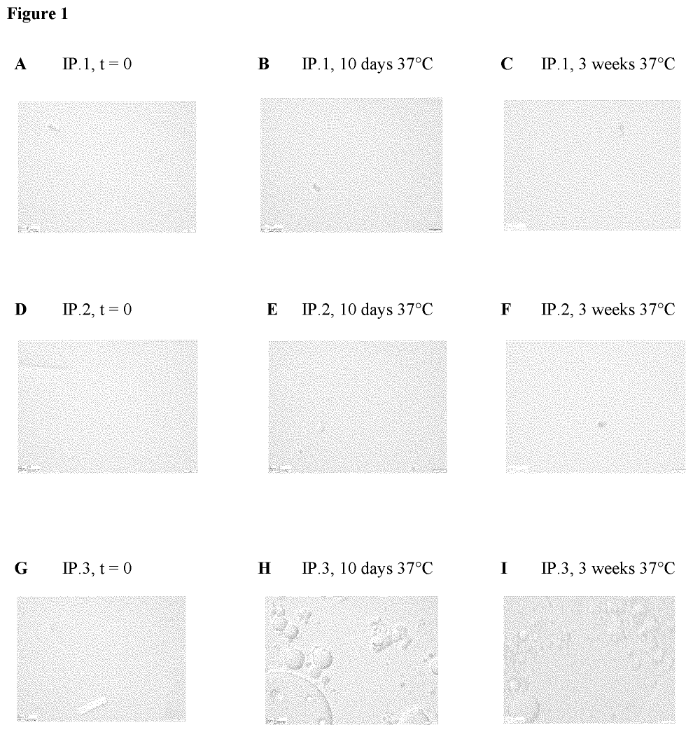

Microscopic views of the samples IP.1, IP.2, and IP.3 are shown in FIG. 1, at t=0; after 10 days at 37° C.; and after 3 weeks at 37° C.

### Mastersizer Results:

| | | | 10 days | 3 weeks at |
|---|---|---|---|---|
| IP | d( ) | t = 0 | 37° C. | 37° C. |
| 1 | 0.1 | 0.07 | 0.07 | 0.07 |
| | 0.5 | 0.11 | 0.11 | 0.11 |
| | 0.9 | 0.16 | 0.16 | 0.16 |
| | 0.99 | 0.21 | 0.22 | 0.21 |
| | 1.00 | 0.27 | 0.28 | 0.27 |
| 2 | 0.1 | 0.07 | 0.07 | 0.07 |
| | 0.5 | 0.11 | 0.11 | 0.11 |
| | 0.9 | 0.16 | 0.16 | 0.16 |
| | 0.99 | 0.22 | 0.21 | 0.21 |
| | 1.00 | 0.29 | 0.27 | 0.27 |
| 3 | 0.1 | 0.08 | 0.13 | 0.15 |
| | 0.5 | 0.13 | 6.48 | 10.54 |
| | 0.9 | 0.19 | 24.68 | 37.65 |
| | 0.99 | 0.24 | 51.35 | 60.80 |
| | 1.00 | 0.30 | 230.27 | 74.34 |

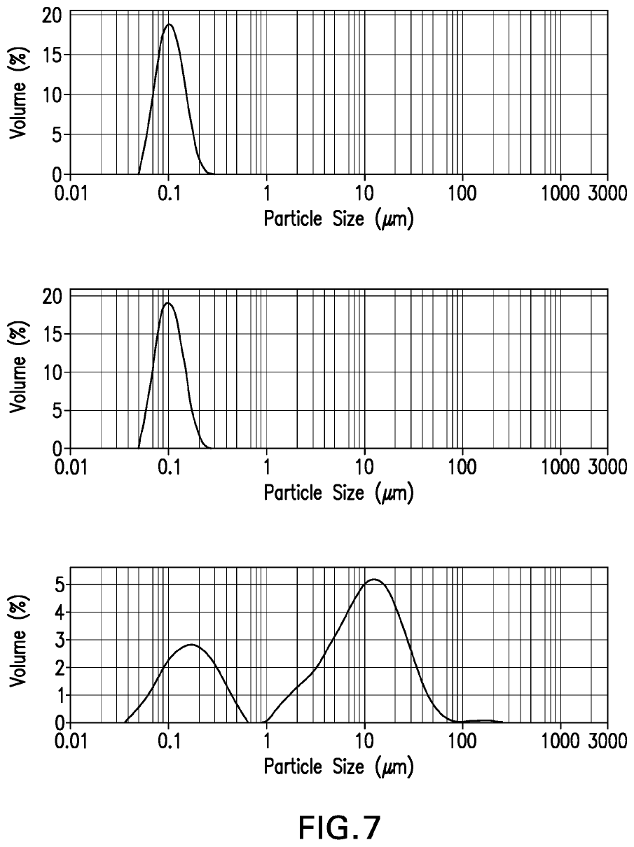

A graphic representation of the Mastersizer results for the three samples after 10 days at 37° C., is provided in FIG. 7.

The results of the test samples reveals breaking of the reference adjuvant composition IP.3, after 10 days at 37° C., and FIG. 7 shows that a large peak appeared which was centred around 10 μm. On the other hand, the adjuvant compositions IP.1 and IP.2, both comprising the SVEA-E double plus adjuvant, maintain an intact emulsion over time.

### Conclusion

Emulsion vaccines comprising a crude Mhyo antigen preparation and comprising an adjuvant composition having Eumulgin B1 as emulsifier, are clearly more stable than the reference emulsion vaccines with the same antigen, but with Polysorbate 80 as the emulsifier. The reference breaks within 10 days at 37° C., while the Eumulgin emulsions do not show any decline in emulsion quality even after 3 weeks at 37° C.

### Example 5: EV0420

The EV0420 is a 2× w/v concentrate, comprising vitamin E-acetate, Shell Ondina X420, and Eumulgin B1. The density is equal to SVEA.

The final vaccine contained the following components and strengths:
Eumulgin B1:1.62% w/v
Ondina X420 (d=0.81 gr/ml): 3.375% w/v
Vitamin E Acetate (0.953 gr/ml): 3.97% w/v
Density of final vaccine=0.9913 g/ml

The O/W emulsion was prepared as a 2× concentrate. WFI was used as water phase. High pressure homogenization was used.

### Composition of the 2× Concentrate:

Per 100 g (101.73 ml):

Oily phase

| | | |
|---|---|---|
| Eumulgin B1 | 3.24 | gr |
| Ondina X420 (Shell) | 6.75 | gr |
| DL-alpha tocopherol acetate | 7.94 | gr |

(corrected for purity)

Water phase

| | |
|---|---|
| WFI | ad 100.00 gr |

### Preparation:

### Oily Phase

1. Weigh the amount of Ondina X420.
2. Add the amount of Vitamin E Acetate and homogenize until it is a clear mixture.
3. Add the amount of Eumulgin B1 and homogenize at 70° C., a clear solution appeared.
4. Heat this mixture up to 70° C.
5. At a temperature of 70° C. sterilize this mixture by filtration using a Nylon66 or PVDF 0.2 μm membrane filter in a filter house provided with a double jacket to keep the temperature at 70° C. during filtration.
6. Cool it down to 55-65° C.

### Water Phase

7. Use sterilized (autoclaved) WFI, heated up to 55-65° C.

### Final Product

8. Prepare premix by adding oily phase to the water phase at high shear mixing.
9. Continue mixing for 5 minutes.

Treat the premix, 3 passages through (sterilized) microfluidizer at 800 bar (temp 55-75° C.).

Microscopically the EV0420 2× w/v concentrate appears as an O/W nano emulsion with some droplets between 1 μm and 5 μm.

### Example 6: Xsolve2.0

The concentrate is based on the Xsolve adjuvant except that the mineral oil was replaced in volume by a Shell Ondina X GTL-based medicinal white oil, and the Polysorbate 80 was replaced in weight by Eumulgin B1

In Xsolve2.0 the ratio of the Microsol-E concentrate:Fortasol-E concentrate=5:1 v/v

### 1. Microsol-E and a Shell Ondina X GTL-Based Medicinal White Oil Concentrate:

Formulation Per 100 ml (Calculated Density 0.8925 g/ml):

| | | |
|---|---|---|
| a Shell Ondina X GTL-based medicinal white oil | 39.25 | g |
| Eumulgin B1 | 2.00 | g |
| WFI | 48.00 | g |

### Preparation:

### Water Phase

1. Weigh the amount of WFI
2. Add the amount of Eumulgin B1 and homogenize at 50° C., a clear solution appeared
3. Sterilize 20 minutes at 121° C. in an autoclave
4. Cool down to 55-65° C.

### Oily Phase

5. Use sterilized (0.2 μm filtered) Shell Ondina X GTL-based medicinal white oil. Heat up to 55-65° C.

### Final Product

6. Prepare premix by adding oily phase to the water phase at high shear mixing
7. Continue mixing for 5 minutes
8. Treat the premix, 3 passages through (sterilized) microfluidizer at 800 bar (temp 45-55° C.)

A micro emulsion having oil droplets with 99%<1 μm should appear

Microscopically the Microsol-E/Shell Ondina X GTL-based medicinal white oil adjuvant composition appears as a homogenous O/W emulsion with some droplets between 1 μm and 5 μm.

### 2. Fortasol-E Concentrate 2×:

Formulation Per 100 ml (Density=1.00 g/ml)

| DL-α-tocopherol acetate | 15.00 | g |
|---|---|---|
| | (corrected for purity) | |
| Eumulgin B1 | 6.00 | g |
| WFI | ad 100.00 | g |

### Preparation:

### Oily Phase

1. Weigh amount of DL-α-tocopherol acetate
2. Add the amount of Eumulgin B1 and homogenize at 50° C., a clear solution appeared
3. Sterilize mixture by 0.2 μm filtration at 50° C.
4. Heat up to 75-85° C.

### Water Phase

5. Use sterile (autoclaved) water for injection, and
6. Heat up to 75-85° C.

### Final Product:

1. Add oily phase slowly to the water phase at 75-85° C. while stirring with visible vortex
2. After addition, continue stirring for 1 minute
3. Check microscopic view, a nano emulsion with few visible particles should appear

Microscopically the Fortasol-E concentrate appears as a homogenous O/W emulsion with some droplets between 1 μm and 5 μm.

### Formulation and Calculated Density of Final Vaccines:

Xsolve50 2.0 Per 100 ml (0.9552 g/ml)

| | | |
|---|---|---|
| Microsol Eumulgin B1/Shell Ondina X | 37.19 | g |
| GTL-based medicinal white oil concentrate: | | |
| Fortasol-E concentrate 2x: | 8.33 | g |
| Antigen + buffer | 50.00 | g |

Xsolve30 2.0 per 100 ml (0.9731 g/ml)

| | | |
|---|---|---|
| Microsol Eumulgin B1/Shell Ondina X | 22.31 | g |
| GTL-based medicinal white oil concentrate: | | |
| Fortasol-E concentrate 2x: | 5.00 | g |
| Antigen + buffer | 70.00 | g |

Xsolve12 2.0 per 100 ml (0.9893 g/ml)

| | | |
|---|---|---|
| Microsol Eumulgin B1/Shell Ondina X | 8.93 | g |
| GTL-based medicinal white oil concentrate: | | |
| Fortasol-E concentrate 2x: | 2.00 | g |
| Antigen + buffer | 88.00 | g |

### Example 7: Stability Study Using Xsolve2.0 (Eumulgin B1/Marcol 52)

In commercial Xsolve oil-in-water concentrate Polysorbate 80 was replaced in weight by Eumulgin B1 to obtain Xsolve2.0 comprising:

| Preparation | Microsol conc. | Fortasol-E conc. | Mhyo antigen |
|---|---|---|---|
| 1 | Polysorbate 80/ | Polysorbate 80 | 50% v/v |
| | Marcol 52 | | |
| 2 | Eumulgin B1/ | Eumulgin B1 | |
| | Marcol 52 | | |

NB: For the purpose of these comparative experiments, these vaccine compositions contained crude Mhyo bacterin antigen, but in an amount that far exceeded the amount present in any comparable commercial Mhyo O/W vaccine; this in order to force the appearance of an effect of the crude antigen on the PS80 emulsifier and correspondingly on the stability of the emulsion.

The above adjuvant composition was subjected to a stability study, wherein appearance (by light microscopic view) and particle sizes (by Mastersizer measurements) were determined.

The appearance of Xsolve2.0 (Eumulgin B1/Marcol 52) after 1 week at 37° C. is homogenous. In contrast thereto, in the reference adjuvant composition (Polysorbate 80/Mineral oil) breaking is observed.

Mastersizer results in μm after 1 week at 37° C.:

| | | | T = 1 week |
|---|---|---|---|
| Adjuvant composition | D | t = 0 | at 37° C. |
| PS80/Mineral oil | 0.1 | 0.17 | 0.20 |
| (reference) | 0.5 | 0.32 | 0.45 |
| | 0.9 | 0.60 | 47.60 |
| | 0.99 | 0.85 | 116.76 |
| | 1.00 | 1.02 | 176.08 |
| Eumulgin B1/Mineral | 0.1 | 0.17 | 0.17 |
| oil | 0.5 | 0.31 | 0.31 |
| | 0.9 | 0.58 | 0.58 |
| | 0.99 | 0.81 | 0.82 |
| | 1.00 | 0.99 | 1.00 |

### Conclusions

Polysorbate 80 can be replaced by Eumulgin B1
Using Eumulgin B1 results in stable emulsions, compared to emulsions using Polysorbate 80, which break.

### Example 8: Efficacy, Safety and Stability of a SVEA-E/FMD Vaccine Composition

### Vaccination-Challenge Experiment

An animal trial in cattle was performed to compare an FMD vaccine composition based on SVEA-E adjuvant and with FMD virus-like particles as antigen (referred to as the SVEA-E/FMD vaccine), with a classic FMD vaccine formulated with Montanide ISA206. To be able to see differences between groups, both vaccines contained a suboptimal but identical dose (i.e. 5 μg per dose) of FMDV Asial/Shamir/ISR/89 VLP antigen.

Cattle were vaccinated intramuscularly (IM) with 2 ml of vaccine in the left side of the neck. Three weeks after vaccination, all animals were challenged with FMDV, strain Asial/Shamir/ISR/89, by intradermolingual (IDL) inoculation. Blood samples were taken at several timepoints post vaccination and post challenge to measure the serological responses during the study. Post challenge, animals were checked under anaesthesia for FMD-specific lesions

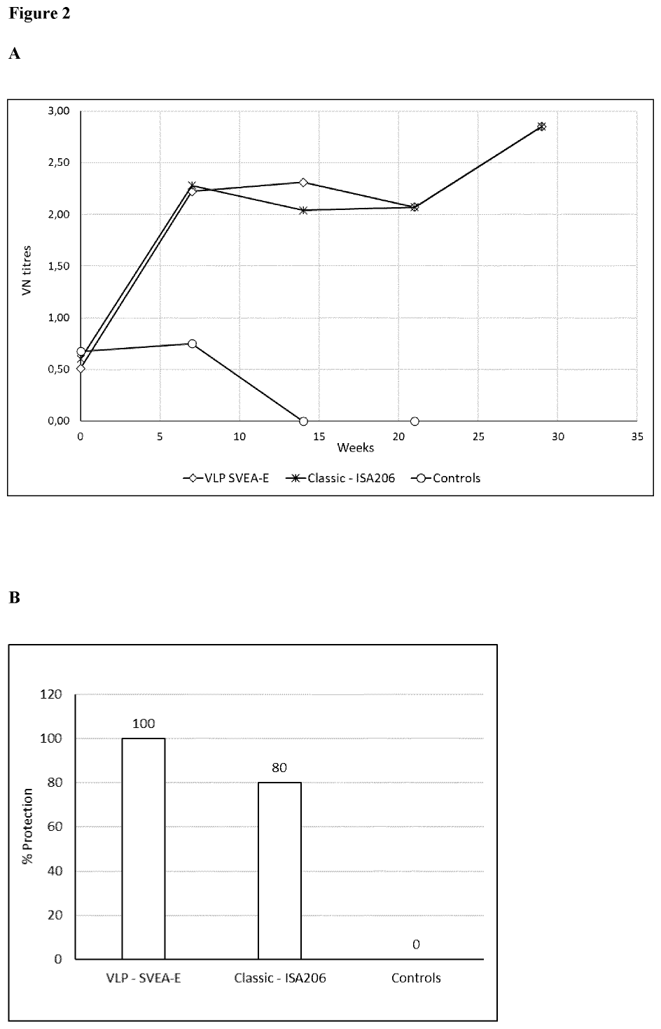

Using the SVEA-E/FMD vaccine composition virus-neutralizing titres were found at the same level of those induced by the classic FMD vaccine (FIG. 2A). The SVEA-E/FMD vaccine provided full protection against challenge, whereas for the classic FMD vaccine 80% protection was observed (FIG. 2B). In contrast to the control animals and the classic ISA206 group, no FMDV-related viremia post challenge was observed in the SVEA-E/FMD group.

Following vaccination, no local reactions were observed in both vaccination groups.

### Duration of Immunity (DOI) in Cattle, Pigs and Goats

A bivalent FMD vaccine was used to immunize the animals, which vaccine contained two types of FMD antigens: VLPs of O/TUR/5/2009 type and of Asial/Shamir/ISR/89 type. Only the virus-neutralizing titres against the O/TUR/5/2009 component were determined.

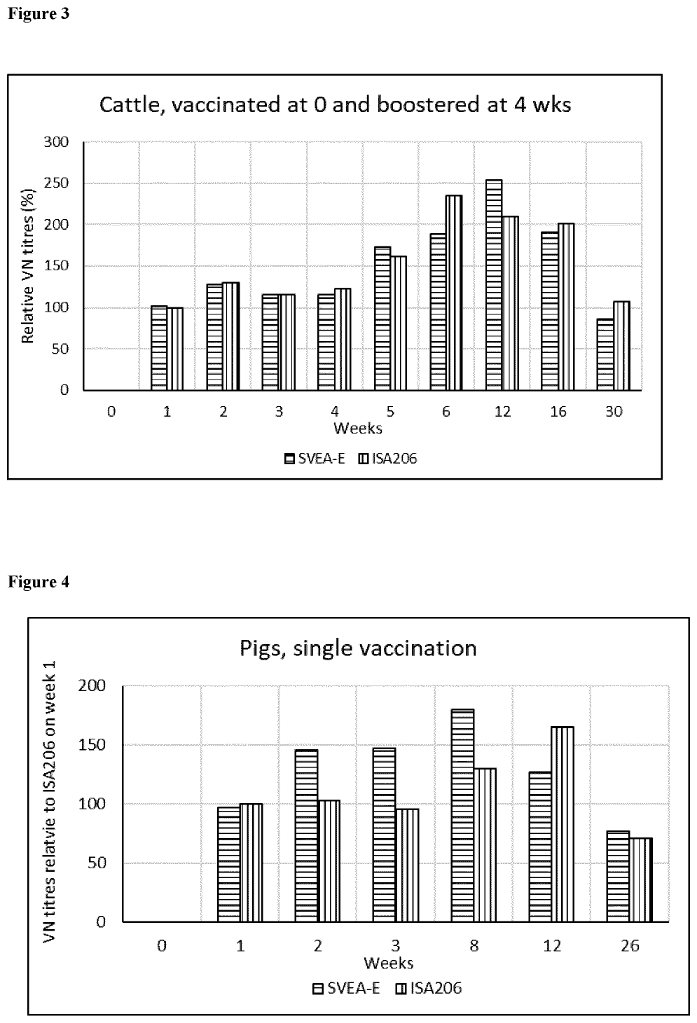

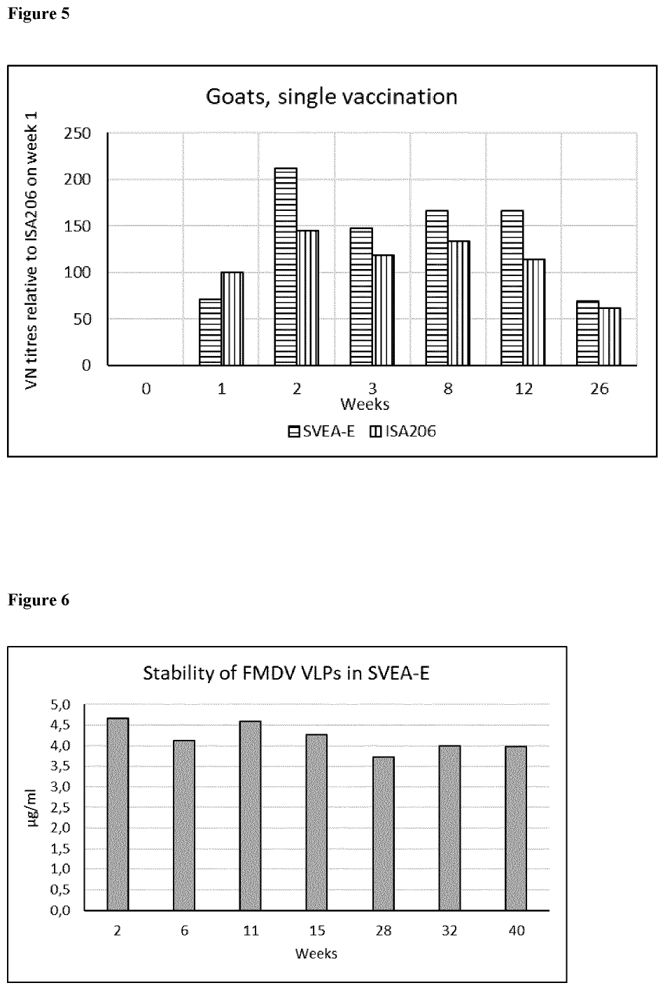

The duration of immunity in cattle (FIG. 3), pigs (FIG. 4) and goats (FIG. 5) using a SVEA-E-adjuvanted FMD vaccine composition, was comparable to the DOI observed from vaccination with a corresponding classic Montanide ISA206-adjuvanted vaccine composition.

Stability of SVEA-E with High Antigen Payload Versus Montanide ISA206

### Compositions:

Vaccine compositions with a mixture of O/TUR/05/2009 and Asia l/Shamir/ISR/89Shm virus-like particles (1:1 v/v)
Antigen solution 15 and 300 (v/v) of the final vaccine.
The density of the antigen solution is considered 1 g/ml, thus the antigen quantities are converted in weight.

Accelerated stability study: visual inspection at 4 and 37° C. 3 weeks stability

| | | | | Oily | Antigen mix | | |
|---|---|---|---|---|---|---|---|
| | | | | phase | concentration | | |
| IP | Antigens | Adjuvant | Emulsifiers | (w/w) | (v/v) | At 4° C. | At 37° C. |
| 1 | O/TUR05 + | W/O | Span80/ | 70% | 15% | sedimented | sedimented |
| 2 | Asia/Shm | emulsion | Tween80 | | 30% | sedimented | sedimented |
| | | adjuvant with | | | | | |
| | | Marcol52 as | | | | | |
| | | liquid | | | | | |
| | | paraffin in a | | | | | |
| | | water/oil | | | | | |
| | | ratio 45/55 | | | | | |
| | | w/w having | | | | | |
| | | less than | | | | | |
| | | 200 mPa · s | | | | | |
| | | viscosity | | | | | |
| 3 | | GNE | Span80/ | 55% | 15% | sedimented | sedimented |
| 4 | | | EumulginB1 | | 30% | sedimented | sedimented |
| 7 | | SVEA | — | | 15% | stable | stable |
| 8 | | | | | 30% | stable | stable |
| 9 | | SVEA-E | | | 15% | stable | stable |
| 10 | | | | | 30% | stable | creamed |
| 11 | | micro | | | 15% | creamed | stable |
| 12 | | SVEA-E | | | 30% | creamed | creamed |
| 13 | | ISA206 | — | 50% | 15% | broken | broken |
| | | | | | 30% | broken | broken |
| 14 | | | | | | | |

From these data it can be concluded that a vaccine composition with a high antigen payload is stable when based on SVEA-E emulsion, but not when based on an Montanide ISA206 emulsion.

NB: The O/TUR/05/2009 and Asial1/Shamir/ISR/89Shm VLP capsid antigens used in this experiment were of pre-development quality, therefore they were relatively crude, as compared to future FMD VLP antigens of a fully developed and optimised commercial product quality. This only makes the stability results even more impressive.

An antigenic mass ELISA can be used to quantify VLPs in the vaccine composition without breaking the emulsion, which is not possible with Montanide ISA206-based formulations. VLPs in vaccines are stable.
Vaccine composition is based on quantification of inactivated FMDV O/TUR/5/2009 VLPs with VHHI-ELISA
Vaccine formulated with SVEA-E adjuvant composition
Strong immune response elicited in cattle after prime—(at 0 dpv) and booster vaccination 42 days later (see results in below table)
After vaccine formulation: same quantification as before formulation (VHHI ELISA)
   no need to break the emulsion vaccine based on SVEA-E
   vaccine stability monitored: stable for >9 months at 4° C. (see FIG. 6)

| | | VN titre | | | | | | |
|---|---|---|---|---|---|---|---|---|
| | | 0 DPV | 7 DPV | 14 DPV | 21 DPV | 28 DPV | 42 DPV | 49 DPV |
| Vaccine | Animal ID | Log10 | Log10 | Log10 | Log10 | Log10 | Log10 | Log10 |
| O/TUR/5/2009 | 1 | n/a | 2.11 | 2.11 | 2.55 | 2.71 | 1.95 | 2.85 |
| VLP (≅8 μg) | 12 | n/a | 2.41 | 2.41 | 3.01 | 2.85 | 2.41 | 3.15 |
| | 3 | n/a | 2.11 | 1.81 | 2.55 | 2.55 | 2.25 | 2.85 |
| | 4 | n/a | 2.41 | 2.25 | 2.41 | 2.55 | 2.25 | 2.85 |
| | 5 | n/a | 1.81 | 2.41 | 2.11 | 2.25 | 1.81 | 2.85 |

### Antigen Information:

### Asial/Shamir/ISR/89

The Asial/Shamir/ISR/89 VLPs (containing capsid-stabilizing mutations in the VP2 protein: S093C and K190N) were produced by way of a baculovirus expression system. In 2-liter bioreactors Tnao38 insect cells were infected at MOI 0.1 with the corresponding recombinant baculovirus. Cell culture supernatant was harvested at 5 dpi by centrifugation, treated with binary ethylenimine (BEI) to inactivate the recombinant baculoviruses, and subsequently concentrated by filtration. Vaccine was formulated with SVEA-E adjuvant composition. The concentration of intact VLPs was determined by ELISA using the M332F antibody (Harmsen et al., 2017, Front. Immunol., vol. 8, p. 960). Serially diluted samples were incubated for 1 h at 37° C. on microtitre plates coated overnight at 4° C. with M332F antibody. After removing the samples and three washes with PBS-Tween, a fixed amount of a biotinylated M332F was added to plates and incubated for 1 h at 37° C. The biotinylated antibody was removed and plates were washed three times with PBS-Tween, after which peroxidase-conjugated streptavidin was added to the plates followed by chromophoric detection.

### O/TUR/5/2009 Virus-Like Particles

Similarly, The O/TUR/5/2009 VLPs (containing a capsid-stabilizing mutation in the VP2 protein: S093C) were produced in 2-litre bioreactors containing Tnao38 insect cells that were infected at an m.o.i. of 1. Cell culture supernatant was harvested at 5 dpi by centrifugation at 200 xg. The concentration of intact VLPs was determined by ELISA using VHH C1 (Wang et al., 2015, BMC Veterinary Research 11:120, DOI 10.1186/si2917-015-0437-2). For this, serially diluted samples were incubated for 1 h at 37° C. on microtiter plates coated overnight at 4° C. with CL. After removing the samples and three washes with PBS-Tween, a fixed amount of a biotinylated C1 was added to plates and incubated for 1 h at 37° C. The biotinylated antibody was removed and plates were washed three times with PBS-Tween, after which peroxidase-conjugated streptavidin was added to the plates followed by chromophoric detection.

### Conclusions

Especially surprising from these results were the findings that:
1. an O/W emulsion type vaccine composition can be used as an FMD vaccine,
2. the vaccine efficacy against an FMDV challenge infection was even better than that obtained using a similar dose of antigen but formulated in the way of the FMDV gold standard type of vaccine: a classic IS0206 W/O/W emulsion vaccine,
3. these vaccines were effective cross-species, and
4. apart from being even more effective than the current standard type of FMD vaccine, the vaccine compositions according to the invention have several important further advantages, most notably for the stability of the emulsion, but also in handling and processing.

1. Adjuvant composition comprising an emulsion of water, a tocopherol or a pharmaceutically acceptable ester thereof, and a polyethoxyethylene cetostearyl ether.

2. Adjuvant composition according to claim 1, wherein the polyethoxyethylene cetostearyl ether is selected from polyethoxyethylene 12 cetostearyl ether, polyethoxyethylene 20 cetostearyl ether and polyethoxyethylene 30 cetostearyl ether.

3. Adjuvant composition according to claim 1 or 2, wherein the polyethoxyethylene cetostearyl ether is polyethoxyethylene 12 cetostearyl ether.

4. Adjuvant composition according to any one of the preceding claims wherein the pharmaceutically acceptable ester of tocopherol is an alpha-tocopheryl acetate.

5. Adjuvant composition according to any one of the preceding claims, further comprising squalane.

6. Adjuvant composition according to any one of the preceding claims, which is free of esters of fatty acids.

7. Adjuvant composition according to any one of the preceding claims, further comprising a non-mineral oil, preferably a synthetic oil.

8. Adjuvant composition according to any one of the preceding claims, wherein the adjuvant composition is an oil-in-water emulsion.

9. Adjuvant composition according to any one of the preceding claims, wherein the polyethoxyethylene cetostearyl ether is present in an amount of about 1 to about 9% by weight based on the volume of the adjuvant composition.

10. Adjuvant composition according to any one of the preceding claims, wherein tocopherol or a pharmaceutically acceptable ester thereof, is present in an amount of about 1 to about 20% by weight based on the volume of the adjuvant composition.

11. Adjuvant composition according any one of claims 5-10, wherein squalane is present in an amount of about 5 to about 20% by weight based on the volume of the adjuvant composition.

12. Adjuvant composition according any one of claims 6-11, wherein the mineral oil is present in an amount of about 5 to about 40% by weight based on the volume of the adjuvant composition.

13. Adjuvant composition according any one of claims 6-12, wherein the non-mineral oil is present in an amount of about 5 to about 40% by weight based on the volume of the adjuvant composition.

14. Adjuvant composition according to any one of the preceding claims, further comprising silica.

15. Adjuvant composition according to any one of the preceding claims comprising emulsion particles having a particle size D50 of about 80 to about 200 nm, preferably of about 40 to about 100 nm.

16. Use of an adjuvant composition according to any one of the preceding claims for formulating an emulsion vaccine, preferably an oil-in-water emulsion vaccine.

17. Vaccine composition comprising the adjuvant composition according to any one of claims 1 to 15, and an antigen.

18. Vaccine composition according to claim 17, wherein the antigen comprises a crude antigen.

19. Vaccine composition according to claim 17 or 18, wherein the antigen comprises an esterase, preferably a lipase.

20. Vaccine composition according to any one of claims 17-19, wherein the vaccine composition comprises one or more antigens selected from a non-live antigen of a bacterium, of a parasite, and of a virus.

21. Vaccine composition according to claim 20, wherein the non-live antigen is from porcine circovirus type 2, Mycoplasma hyopneumoniae, and/or foot-and-mouth disease virus.

22. Vaccine composition according to any one of claims 17-21, wherein the vaccine composition is an emulsion vaccine.

23. Vaccine composition according to claim 22, wherein the emulsion vaccine is an oil-in-water emulsion vaccine.

24. Method for the manufacture of a vaccine composition according to any one of claims 17-23, the method comprising the steps of:
preparing an aqueous phase comprising an antigen, and
admixing said aqueous phase with the adjuvant composition of any one of claims 1 to 15.

25. Vaccine composition according to any one of claims 17 to 23 for use in a method of protecting a human- or animal target against infection and/or disease caused by a pathogen.

26. Vaccine composition for use according to claim 25, wherein the animal target is a swine or a ruminant.

27. Vaccine composition for use according to claim 25 or 26, wherein the pathogen is selected from a bacterium, a parasite, and a virus.

28. Vaccine composition for use according to any one of claims 25 to 27, wherein the pathogen is selected from one or more of porcine circovirus type 2, Mycoplasma hyopneumoniae, and foot-and-mouth-disease virus.

29. Vaccine composition for use according to any one of claims 25-28, wherein the vaccine composition is administered by intradermal or intramuscular route.

30. Vaccine composition according to any one of claims 17 to 23, for use in the vaccination of a human- or animal target against infection and/or disease caused by a pathogen.

31. Use of the adjuvant composition of any one of claims 1 to 15 for the manufacture of a vaccine composition for protecting a human- or animal target against infection and/or disease caused by a pathogen.

32. Method for the vaccination of a human- or animal target against infection and/or disease caused by a pathogen, the method comprising the administration to said target of the vaccine composition according to any one of claims 17-23.

33. Kit of parts comprising at least two containers, one container comprising the adjuvant composition of any one of claims 1 to 15, and one container comprising an antigen.

34. Kit of parts according to claim 33, wherein the antigen is a freeze-dried live PRRSV, or is a freeze-dried preparation of non-live Lawsonia intracellularis.
